SEQUENCE LISTING (24 sequences)

SEQ ID NO: 1 - DNA, 21 bp, Artificial Sequence
  synthetic construct
ggaattaattgcatgaattag

SEQ ID NO: 2 - DNA, 21 bp, Artificial Sequence
  synthetic construct
gccttgaacctcagctttaag

SEQ ID NO: 3 - DNA, 20 bp, Artificial Sequence
  primer
tgttaccaactgggacgaca

SEQ ID NO: 4 - DNA, 20 bp, Artificial Sequence
  primer
ctatgggagaacggcagaag

SEQ ID NO: 5 - DNA, 23 bp, Artificial Sequence
  primer
aacatacgaaaaacacacccatt

SEQ ID NO: 6 - DNA, 24 bp, Artificial Sequence
  primer
agtgtatggctaagaaaagacctg

SEQ ID NO: 7 - DNA, 20 bp, Artificial Sequence
  primer
cagaccccaggtggtaccgg

SEQ ID NO: 8 - DNA, 20 bp, Artificial Sequence
  primer
taacagatgcacgggggttg

SEQ ID NO: 9 - DNA, 20 bp, Artificial Sequence
  primer
cttccctgttgagttggccc

SEQ ID NO: 10 - DNA, 20 bp, Artificial Sequence
  primer
caagctggagctaaataagc

SEQ ID NO: 11 - DNA, 22 bp, Artificial Sequence
  primer
atcccagttcacttctctcctg

SEQ ID NO: 12 - DNA, 21 bp, Artificial Sequence
  primer
gcaaactagaaggccttagag

SEQ ID NO: 13 - DNA, 24 bp, Artificial Sequence
  primer
ctaatctggtatcccccgagtcac

SEQ ID NO: 14 - DNA, 24 bp, Artificial Sequence
  primer
ccgggacacttttacatacatttg

SEQ ID NO: 15 - DNA, 25 bp, Artificial Sequence
  primer
ccctgctcacataataactcaaatc

SEQ ID NO: 16 - DNA, 22 bp, Artificial Sequence
  primer
ggggctacttggaaaccatttc

SEQ ID NO: 17 - DNA, 26 bp, Artificial Sequence
  primer
caatggaaagcgattatagcaacaag

SEQ ID NO: 18 - DNA, 23 bp, Artificial Sequence
  primer
ggacttcgcaggctcaggcatag

SEQ ID NO: 19 - DNA, 21 bp, Artificial Sequence
  primer
aaccacacccacaggatcaga

SEQ ID NO: 20 - DNA, 22 bp, Artificial Sequence
  primer
tcttcgctttattgctccatga

SEQ ID NO: 21 - DNA, 17 bp, Artificial Sequence
  primer
tgctccccgggctgtat

SEQ ID NO: 22 - DNA, 24 bp, Artificial Sequence
  primer
cataggagtccttctgacccattc

SEQ ID NO: 23 - DNA, 22 bp, Artificial Sequence
  primer
tgctccagcatcgaatttgtac

SEQ ID NO: 24 - DNA, 25 bp, Artificial Sequence
  primer
agagcagctgctgctgcatacactt

(19) United States
(12) Patent Application Publication
(10) Pub. No.: US 2012/0135399 A1
(43) Pub. Date: May 31, 2012

(54) CANCER BIOMARKER AND METHODS OF USING THEREOF

(75) Inventors: Dean Tantin, Salt Lake City, UT (US); Arvind Shakya, Salt Lake City, UT (US)

(73) Assignee: UNIVERSITY OF UTAH RESEARCH FOUNDATION, Salt Lake City, UT (US)

(21) Appl. No.: 13/132,336

(22) PCT Filed: Nov. 24, 2009
(86) PCT No.: PCT/US09/65742

§ 371 Date: Jul. 18, 2011

Related U.S. Application Data
(63) Continuation of application No. 61/200,719, filed on Dec. 3, 2008.
Application No. 61/200,719 is a continuation of application No. 61/245,008, filed on Sep. 23, 2009.

(51) Int. Cl. G01N 33/566; C12Q 1/68; C12Q 1/02

(52) U.S. Cl. 435/6.1; 435/7.1; 435/29

(57) ABSTRACT

Described herein are biomarkers which can be used for identifying a subject at risk for or evaluating the progression of cancer. In certain aspects, these biomarkers can be used to identify cancer stem cells. These biomarkers can include, but are not limited to, Oc1 or molecular variants thereof, Oc1 target proteins, or a combination thereof. In addition, described herein are methods for reducing the expression of these biomarkers associated with cancer.

### CROSS-REFERENCE TO RELATED APPLICATIONS

This application claims benefit of U.S. provisional application Ser. No. 61/200,719, filed Dec. 3, 2008, and U.S. provisional application Ser. No. 61/245,008, filed Sep. 23, 2009, which are hereby incorporated by reference in their entireties for all of their teachings.

### BACKGROUND

During the past fifty years, great strides have been made in cancer diagnostics, treatments, and therapies. These diagnostics and treatments have extended patient's life spans; however, even with the most successful treatments relapse of cancer is highly probable. In addition, for some forms of cancer, there remains no effective treatment options.

Studies of cancer cells have focused on key hallmarks, including the constitutive activation of cell division pathways and suppression of apoptosis. Accordingly, if cell division cascades are constitutively activated or apoptosis is suppressed, cell proliferation occurs thus potentially leading to cancer. Therefore, these pathways have been deemed of great importance.

However, other molecular pathways and phenomena may prove to be viable areas for cancer research. For example, a phenomenon has been observed in which cancer cells have decreased aerobic capacity and increased glycolysis. Stated differently, cancer cells frequently demonstrate increased glycolytic metabolism and decreased oxidative metabolism when compared to normal cells. This phenomenon has been termed the “Warburg effect.”

In addition, tumor ontogeny has proven to be an interesting subject in itself. One theory, suggests that cancer stem cells are a population of cells that gives rise to the bulk of a malignancy's biomass or is the seed for the tumor. A related term is “tumor initiating cell” (TIC); TICs are able to effectively establish a tumor in a congenic animal, or in an immunocompromised mouse for example. In models discussing cancer stem cells, it has been suggested that standard cancer treatments (i.e., chemotherapy, radiation, etc.) often kill a majority of cancer cells but fail to kill the cancer stemline or the cancer stem cells. Therefore, the cancer stemline persists and subsequently produces new cancer cells. To further complicate the diagnosis and treatment of cancer, cancer stem cells are often undetectable. Thus, even after treating many cancers with standard cancer treatments (i.e., chemotherapy, radiation, etc.), cancer often recurs because cancer stem cells remain.

This application focuses on the role of the transcription factor Oct1 and Oct1 target proteins, in tumor ontogeny and their role as a cancer and cancer stem cell biomarker.

### BRIEF SUMMARY

Described herein are biomarkers which can be used for identifying a subject at risk for or evaluating the progression of cancer. In certain aspects, these biomarkers can be used to identify cancer stem cells. These biomarkers can include Oct1 or molecular variants thereof and downstream targets of Oct1. In addition, described herein are methods for reducing the expression of these biomarkers associated with cancer.

Additional advantages of the disclosed composition(s) and method(s) will be set forth in part in the description which follows, and in part will be understood from the description, or may be learned by practice of the disclosed composition(s) and method(s). The advantages of the disclosed composition(s) and method(s) will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed methods and compositions and together with the description, serve to explain the principles of the disclosed methods and compositions.

FIG. 1(a) shows western blotting analysis of primary human metastatic breast carcinoma cells (pleural effusions); FIG. 1(b) and FIG. 1(c) show flow cytometry assays of the pleural effusions.

FIG. 2 shows Oct1 RNAi diminishes the Aldefluor^{Hi} population in human tumor cell lines. FIG. 2(a), FIG. 2(c), and FIG. 2(e) shows flow cytometry analysis in which Oct1-specific RNAi significantly reduced the number of Aldefluor^{Hi} events relative to a scramble control in MB-MDA 231, MCF-7, and A549 cells respectively. FIG. 2(b), FIG. 2(d), and FIG. 2(f) show confirmation of effective RNAi via Western blotting using the MB-MDA 231, MCF-7, and A549 cells respectively. FIG. 2(g) shows a conserved Oct1-binding site in the immediate promoter region of the Aldh1a1 promoter in several example vertebrate species. The conserved perfect octamer sequence centered at approximately −55 bp relative to the transcription start site is highlighted using brackets. FIG. 2(h) shows ChIP assays using MB-MDA 231 cells and Oct1-specific antibodies.

FIG. 3 shows that Oct1 RNAi diminishes the stem cell population of A549 cells in a side-population assay. GFP-positive, luciferase-positive A549 cells carrying an inducible Oct1-specific shRNA were used.

FIG. 4 shows immunofluorescence microscopy images of normal and malignant human colon and breast tissue. Sections were stained with either DAPI or TO-PRO to reveal nuclei, as well as anti-Oct1 and anti-ALDH1a1 antibodies. Merged images are shown on the right. Examples of cells co-staining with Oct1 and ALDH1 are shown with yellow arrows. Examples of cells staining with ALDH1 are shown with asterisks. FIG. 4(a) shows normal human colon saggital sections. FIG. 4(b) shows coronal sections. FIG. 4(c) shows section of a well-differentiated adenocarcinoma from the ascending colon of a 45 year old male patient. FIG. 4(d) shows stage 4 malignant breast carcinoma.

FIG. 5 shows cells in the colon crypt expressing high Oct1 protein levels also express high levels of the stem cell marker ALDH1a.

FIGS. 6(a-f) show images of wild-type (WT) MEFs cultured either in high glucose (a) or glucose-free (b) medium; Oct1^{-/-} MEFs in high glucose (c) or glucose-free (d) medium; Oct1^{-/-} MEFs with ectopic expression of Oct1 in high glucose (e) or glucose-free (f) medium. FIG. 6(g) shows intracellular ATP content in WT and Oct1^{-/-} MEFs (mean±s.e.m., n=3); FIG. 6(h) shows NAD⁺/NADH ratio in WT and Oct1^{-/-} MEFs (mean±s.e.m., n=3); FIG. 6(i) and FIG. 6(j) show rate of oxygen consumption in WT and Oct1^{-/-} MEFs (mean±s.e.m., n=3) and embryos (mean, n=2, j) in the presence and absence of 2,4-dinitrophenol (DNP); FIG. 6(k) shows flow cytometric analysis of WT and Oct1^{-/-} MEFs mitochondrial membrane potential (mean±s.e.m., n=5). RFU=relative fluorescent units.

FIG. 7 shows partial least squares projections to latent structures (PLS) analysis of metabolomics data of the metabolic profiles of Oct1^{-/-} and wild type MEFs.

FIG. 8 shows the intracellular steady state levels of glucose in Oct1^{-/-} and WT MEFs.

FIG. 9 shows the intracellular steady state levels of lactate in Oct1^{-/-} and WT MEFs.

FIG. 10 shows the intracellular steady state levels of tricarboxylic acid cycle (TCA) intermediates in Oct1^{-/-} and WT MEFs.

FIG. 11 shows the intracellular levels of free fatty acids in Oct1^{-/-} and WT MEFs.

FIG. 12 shows the intracellular steady state levels of significantly altered amino acids in Oct1^{-/-} MEFs when compared to WT MEFs.

FIG. 13 shows the intracellular steady state levels of proline and glycine in Oct1^{-/-} MEFs when compared to WT MEFs.

FIG. 14 shows GC/MS data for creatinine and urea, products of amino acid metabolism where urea is increased in Oct1^{-/-} MEFs when compared to WT MEFs.

FIG. 15 shows the rate of glucose oxidation in Oct1^{-/-} MEFs when compared to WT MEFs.

FIG. 16 shows the rate of palmitate oxidation in Oct1^{-/-} MEFs when compared to WT MEFs.

FIG. 17 shows the rate of glutamate oxidation in Oct1^{-/-} MEFs when compared to WT MEFs.

FIG. 18 shows the rate of heat production of WT mice and Oct1^{+/-} mice fed normal chow and fat chow respectively.

FIG. 19 shows the rate of oxygen consumption of WT mice and Oct1^{+/-} mice fed normal chow and fat chow respectively.

FIG. 20 shows the level of physical activity of WT mice and Oct1^{+/-} mice fed normal chow and fat chow respectively.

FIG. 21 shows the metabolic rate of WT mice and Oct1^{+/-} mice fed normal chow and fat chow respectively.

FIG. 22 shows flow cytometric measurements of reactive oxygen species (ROS) production in WT or Oct1^{-/-} lymphocytes adoptively transferred into sub-lethally irradiated rag1^{-/-} mice.

FIG. 23 shows lactate levels of splenic white blood cells from rag1^{-/-} mice repopulated with WT or Oct1^{-/-} fetal liver cells (mean±s.e.m., n=3).

FIG. 24 shows western blot analysis of Oct1 expression in A549 cells harboring doxycycline-inducible shRNAs.

FIG. 25 shows ATP levels in A549 cells expressing control and Oct1 shRNA.

FIG. 26 shows lactate levels in A549 cells expressing control and Oct1 shRNA.

FIG. 27 shows metabolic changes associated with Oct1 overepxression in A549 cells transiently transfected with a control plasmid or a plasmid that expressed Oct1.

FIG. 28 shows a gene expression profile which identifies changes in metabolic gene expression.

FIG. 29 shows Western blotting analysis of pyruvate carboxylase (PCX) and pyruvate dehydrogenase kinase 4 (PDK4) levels in Oct1^{-/-} and WT MEF cells.

FIG. 30 shows expression profiling identifying the changes in amino acid metabolism gene signatures.

FIG. 31 shows ChIP analysis evaluating potential downstream targets of Oct1.

FIG. 32 shows relative PGC-1α mRNA levels determined by quantitative PCR.

FIG. 33 shows Western blotting analysis quantifying the amount of PGC-1α in WT and Oct1^{-/-} MEFs.

FIG. 34 shows a TEM image (×3,900) of the mitochondrial density in WT MEFs.

FIG. 35 shows a TEM image (×3,900) of the mitochondrial density in Oct1^{-/-} MEFs.

FIG. 36 shows a quantification of the mitochondrial DNA present in WT and Oct1^{-/-} MEFs.

FIG. 37 shows anchorage independent colony formation on soft agar using WT, Oct1^{-/-}, p53^{-/-}, p53^{-/-}; Oct1^{+/-}, and p53^{-/-}; Oct1^{-/-} MEFs transformed with H-Ras^{V12}-GFP virus.

FIG. 38 shows quantification of colony number of colonies formed on soft agar using WT, Oct1^{-/-}, p53^{-/-} and Oct1^{+/+}, p53^{-/-}; Oct1^{+/-}, and p53^{-/-}; Oct1^{-/-} MEFs transformed with H-Ras^{V12}-GFP virus.

FIG. 39 shows quantification of colony size of the colonies formed on soft agar using WT, Oct1^{-/-}, p53^{-/-} and Oct1^{+/±}, p53^{-/-}; Oct1^{+/-}, and p53^{-/-}; Oct1^{-/-} MEFs transformed with H-Ras^{V12}-GFP virus.

FIG. 40 shows survival rates of Oct1^{+/-}, p53^{-/-} mice compared to p53^{-/-} mice. C57BL/6 were used in this experiment.

FIG. 41 shows the types of cancers occurring in Oct1^{+/-}, p53^{-/-} and p53^{-/-} mice. C57BL/6 were used in this experiment.

FIG. 42 shows survival rates of Oct1^{+/-}, p53^{-/-} mice compared to p53^{-/-} mice. 129sv mice were used in this experiment.

FIG. 43 shows the types of cancers occurring in Oct1^{+/-}, p53^{-/-} and p53^{-/-} mice. 129sv mice were used in this experiment.

FIG. 44 shows a Kaplan-Meier plot assessing survival rates of liver cells from p53−/− and Oct1−/−; p53−/− embryos were transplanted into irradiated rag1−/− mice.

FIG. 45 shows luciferase expression in A549 cells expressing either scrambled or Oct1 shRNA that were inoculated into nude mice.

FIG. 46 shows the growth rates of A549 cell lines which have been transfected with Oct1 shRNA or scrambled shRNA.

FIG. 47 shows the colony number of p53^{-/-} and p53^{-/-}; Oct1^{+/-} MEFs treat with dichloroacetate (DCA).

FIG. 48 shows the colony size of p53^{-/-} and p53^{-/-}; Oct1^{+/-} MEFs treat with dichloroacetate (DCA).

FIG. 49(a) shows Quantitative RT-PCR showing Chd1 transcript levels in A549 cells carrying inducible scrambled and Oct1-specific siRNAs. P-value was calculated using a two-tailed student T-test. FIG. 49(b) shows example of Oct1 siRNA knockdown. Scrambled siRNAs were used as a control.

FIG. 50 shows a custom antibody directed against Oct1-phospho S335 used to stain mitotic figures in HeLa cells. Alpha-tubulin and DAPI are used as controls. Note the exclusion from DNA and centrosome/spindle pole body/midbody staining

FIG. 51 shows Oct1 RNAi applied to HeLa cells (which have inactive p53 due to the presence of HPV E6 protein) results in abnormal mitoses. FIG. 51(a) shows effect of siRNA on Prophase and Metaphase. FIG. 51(b) shows effect of siRNA on Anaphase and Telophase. These results were not duplicated in A549 lung adenocarcinoma cells, which have intact p53.

### DETAILED DESCRIPTION

The disclosed methods and compositions may be understood more readily by reference to the following detailed description of particular embodiments and the Example included therein and to the Figures and their previous and following description.

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

Disclosed are materials, compositions, and components that can be used for, can be used in conjunction with, can be used in preparation for, or are products of the disclosed composition(s) and method(s). These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. Thus, if a class of molecules A, B, and C is disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F is specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed, it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the composition(s) and method(s) described herein. Such equivalents are intended to be encompassed by the appended claims.

It is understood that the disclosed composition(s) and method(s) are not limited to the particular methodology, protocols, and reagents described as these may vary. It is also to be understood that the terminology used herein is for the purpose of describing particular embodiments only and is not intended to limit the scope of the present invention which will be limited only by the appended claims.

Unless defined otherwise, all technical and scientific terms used herein have the same meanings as commonly understood by one of skill in the art to which the disclosed methods and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents.

In this specification and in the claims that follow, reference will be made to a number of terms that shall be defined to have the following meanings:

It must be noted that, as used in the specification and the appended claims, the singular forms “a,” “an,” and “the” include plural referents unless the context clearly dictates otherwise. Thus, for example, reference to “a metabolic factor” includes mixtures of two or more such metabolic factors, and the like.

Throughout the description and claims of this specification, the word “comprise” and variations of the word, such as “comprising” and “comprises,” means “including but not limited to” and is not intended to exclude, for example, other additives, components, integers or steps.

“Optional” or “optionally” means that the subsequently described event or circumstance can or cannot occur, and that the description includes instances where the event or circumstance occurs and instances where it does not. For example, the phrase “optionally a therapeutic agent” means that the therapeutic agent can or can not be included.

“Subject” refers to mammals including, but not limited to, humans, non-human primates, sheep, dogs, rodents (e.g., mouse, rat, etc.), guinea pigs, cats, rabbits, cows, and non-mammals including chickens, amphibians, and reptiles, who are at risk for or have been diagnosed with cancer and benefits from the methods and compositions described herein

“Biological Sample” refers to cells and/or tissues obtained from a biopsy sample, a surgical resection, blood, plasma, serum, urine, stool, spinal fluid, nipple aspirates, lymph fluid, external secretions of the skin, respiratory tract, intestinal and genitourinary tracts, bile, saliva, milk, tumors, organs, cancer tissue, a tissue sample, primary ascites cells and in vitro cell culture constituents.

“Cancer Stem Cells” refers to a small percentage of progenitor cells (or tumor initiating cells), located near or within a tumor, that are typically resistant to traditional cancer therapies (i.e., chemotherapy and radiation therapy), capable of self-renewal, and capable of regenerating a tumor. CSCs can generate tumors through the stem cell processes of self-renewal and differentiation into multiple cell types.

“Oct1 Target Protein” refers to a protein that is either upregulated or down regulated by Oct1.

“Oxidative Metabolism” or cellular respiration, refers to an oxygen dependent cellular process occurring within the mitochondria of a cell in which various metabolic factors including, but not limited to, nicotinamide adenine dinucleotide (NADH), nicotinamide adenine dinucleotide phosphate (NADPH), adenosine triphosphate (ATP), and carbon dioxide (CO₂) are formed. Also, during oxidative metabolism, oxygen is consumed as the terminal electron acceptor, generating water.

“Glycolytic Metabolism” refers to a cellular process occurring in the cytoplasm of a cell in which glucose is broken down to yield various metabolic factors including, but not limited to, ATP and lactate. During glycolytic metabolism, oxygen is NOT consumed.

“Metabolic Factor” includes products of both glycolytic and oxidative metabolism. Examples of metabolic factors include, but are not limited to, intracellular lactate, intracellular oxygen consumption, intracellular ATP production, intracellular NAD+/NADH ratio, intracellular glucose, intracellular glucose oxidation, intracellular palmitate oxidation, intracellular glutamate oxidation, and intracellular amino acids.

The term “nucleic acid” may be used to refer to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

When describing variants in proteins or peptides, the term “variant” refers to an amino acid or peptide sequence having conservative amino acid substitutions, non-conservative amino acid substitutions (i.e. a degenerate variant), substitutions within the wobble position of each codon (i.e. DNA and RNA) encoding an amino acid, amino acids added to the C-terminus of a peptide, or a peptide having 60%, 65%, 70%, 75%, 80%, 85%, 90%, 95%, 96%, 97%, 98%, or 99% homology to a reference sequence.

When describing variants in nucleic acid sequences, the term “variant” refers to a substitution, an insertion, a deletion, or a combination thereof of one or more nucleotides within a nucleic acid sequence. Theses substitutions, insertions, deletions, or a combination thereof can result in a nonsense mutation, a missense mutation, a frameshift mutation, a silent mutation, or a neutral mutation. When describing variants in nucleic acid sequences, variant can include a sequence with 60%, 65%, 70%, 75%, 80%, 85%, 90%, 95%, 96%, 97%, 98%, or 99% homology when compared to the reference sequence.

The terms “homology”, “identity” and “similarity” refer to the degree of sequence similarity between two peptides or between two optimally aligned nucleic acid molecules. Homology and identity can each be determined by comparing a position in each sequence which can be aligned for purposes of comparison. For example, it is based upon using a standard homology software in the default position, such as BLAST, version 2.2.14. When an equivalent position in the compared sequences is occupied by the same base or amino acid, then the molecules are identical at that position; when the equivalent site occupied by similar amino acid residues (e.g., similar in steric and/or electronic nature such as, for example conservative amino acid substitutions), then the molecules can be referred to as homologous (similar) at that position. Expression as a percentage of homology/similarity or identity refers to a function of the number of similar or identical amino acids at positions shared by the compared sequences, respectfully. A sequence which is “unrelated” or “non-homologous” shares less than 40% identity, though preferably less than 25% identity with the sequences as disclosed herein.

As used herein, the term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T. C, G. U. or I) or residue occurs in both sequences to yield the number of matched positions, dividing the number of matched positions by the total number of positions in the comparison window (i.e., the window size), and multiplying the result by 100 to yield the percentage of sequence identity

The term “expression” as used herein refers to the transcription of a nucleic acid sequence (i.e., RNA), as well as to the production, by translation, of a polypeptide product from a transcribed nucleic acid sequence.

As used herein, detection of the “levels” of a given analyte can refer to either quantitative or qualitative modes of detection of the analyte. These methods do not require, but can include, measurements of the levels.

By a “decrease”, “reduction” or “inhibition” used in the context of the level of expression or activity of a gene refers to a reduction in protein or nucleic acid level. For example, such a decrease may be due to reduced RNA stability, transcription, or translation, increased protein degradation, or RNA interference. Preferably, this decrease is at least about 5%, at least about 10%, at least about 25%, or when “decrease” is used in the context of a decrease the expression of a cancer stem cell biomarker as compared to a reference expression level, a decrease is preferably at least about 50%, at least about 60%, at least about 70%, at least about 80%, at least about 90%, or at least 100% (i.e. complete inhibition), or any integer in between of the level of expression or activity under control conditions (i.e. normal expression levels).

By an “increase” in the expression or activity of a gene or protein is meant a positive change in protein or nucleic acid level. For example, such an increase may be due to increased RNA stability, transcription, or translation, or decreased protein degradation. Preferably, this increase is at least 5%, at least about 10%, at least about 25%, at least about 50%, at least about 75%, at least about 80%, at least about 100%, or when “increase” is used in the context of an increase in the expression of a cancer stem cell biomarker as compared to a reference expression level, an increase is preferably at least about 150% (i.e. 1.5-fold), at least about 200% (i.e. 2-fold), or at least about 300% (i.e. 3-fold) or at least about 500% (i.e. 5-fold), or at least about 1,000% (i.e. 10-fold) or more over the level of expression or activity under control conditions.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within the ranges as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. as well as 1, 2, 3, 4, and 5, individually. The same principle applies to ranges reciting only one numerical value as a minimum or a maximum. Furthermore, such an interpretation should apply regardless of the breadth of the range or the characteristics being described.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### A. METHODS

There herein disclosed compositions and methods relate generally to the expression and activity of Oct1 by cancer stem cells.

Oct1 has traditionally been classified as a member of the POU (Pit-1, Oct1/2, Unc-86) domain family of transcription factors. In addition, although not overtly transforming (in the same sense as Myc and Ras for example), Oct1 overexpression can be pro-tumorigenic. For illustrative purposes, non-limiting examples of human Oct1 and known polymorphs are included within Table 1.

TABLE 1
Oct1 polymorphs

| dbSNP entry | Position (Build HG18) | change | Outcome |
|---|---|---|---|
| rs1136938 | chr1: 165619742-165619742 | T→A | missense |
| | | | (L→Q) |
| rs12030882 | chr1: 165635101-165635101 | G→A | missense |
| | | | (E→K) |
| rs34379394 | chr1: 165651603-165651603 | T→C | missense |
| | | | (S→P) |
| rs72057527 | chr1: 165625587-165625587 | 1 bp | Frameshift |
| | | delete (−T) | |
| rs34899926 | chr1: 165606030-165606030 | C→T | coding |
| | | | synonymous |
| rs41270710 | chr1: 165606072-165606072 | A→G | coding |
| | | | synonymous |
| rs34958084 | chr1: 165619723-165619723 | C→T | coding |
| | | | synonymous |
| rs7534943 | chr1: 165647854-165647854 | C→T | coding |
| | | | synonymous |
| rs2229284 | chr1: 165647875-165647875 | A→G | coding |
| | | | synonymous |
| rs34658638 | chr1: 165651431-165651431 | G→A | coding |
| | | | synonymous |

For these SNPs references the Oct1/Pou2f1 RefSeq ID NM_002697

Oct1 plays a crucial role in intracellular metabolism. As disclosed herein, compared to cells expressing “normal, physiological” levels of Oct1, overexpression of Oct1 can shift intracellular metabolism from oxidative metabolism to glycolytic metabolism, and underexpression of Oct1 can further shift intracellular metabolism to favor a greater level of oxidative metabolism respectively.

In some aspects, cancer cells, such as cancer stem cells, overexpress Oct1 or a molecular variant thereof. In this aspect, intracellular metabolism shifts from “oxidative metabolism” to “glycolytic metabolism” when Oct1 or a molecular variant thereof is overexpressed and can have a tumorigenic effect. In contrast, underexpression of Oct1 can lead to a change in intracellular metabolism that can be linked to anti-tumorigenicity. As disclosed herein, Oct1-deficient cells and cells overexpressing Oct1 both demonstrate altered cellular metabolism. For example, Oct1-deficient cells demonstrated augmented mitochondrial function: increased oxygen consumption, TCA intermediates, ETC complexes, PGC-1α levels, and mitochondrial genome content, density, and membrane potential. In contrast, cells overexpressing Oct1 demonstrated the opposite results.

Thus, as disclosed herein, the amount of one or more various metabolic factors associated with oxidative metabolism changes when a shift from oxidative metabolism to glycolytic metabolism occurs, and intracellular metabolism can be altered. In some aspects, these metabolic factors include, but are not limited to, intracellular lactate, intracellular oxygen consumption, intracellular ATP production, intracellular NAD+/NADH ratio, mitochondrial membrane potential, intracellular glucose, intracellular glucose oxidation, intracellular palmitate oxidation, intracellular glutamate oxidation. In some aspects, the metabolic factors include, but are not limited to, Oct1 Target Proteins (OTP). In this aspect, intracellular gene expression of these OTPs is affected. As disclosed herein, OTPs can include, but are not limited to, pyruvate carboxylase (PCX), pyruvate dehydrogenase kinase 4 (PDK4), dihyrdolipoamide acetyltransferase (Dlat), isocitrate dehydrogenase, succinate dehydrogenase, peroxisome proliferator-activated receptor-γ co-activator-1 alpha (PGC-1α), glutaminyl tRNA synthetase, glutamate-cysteine ligase, phosphoribosyl pyrophosphate amidotransferase, cabamoyl-phosphate synthetase 2, aspartate transcabamylase, dihydroorotase, glutathione reductase 1, adenylosuccinate lyase 1, or any combination thereof.

In certain aspects, many of the OTPs listed above play pivotal roles either in glycolytic metabolism or oxidative metabolism, and a change in any of these OTPs by either an upregulation or a downregulation can result in a subsequent metabolic shift. For example, intracellular glycolytic metabolism or oxidative metabolism can either increase or decrease depending on which OTP is either upregulated or down-regulated.

Pyruvate carboxylase (PCX) is an enzyme of the ligase class that catalyzes irreversible carboxylation of pyruvate to form oxaloacetate (OAA). Furthermore this enzyme catalyzes an anaplerotic reaction that provides an oxaloacetate precursor for the citric acid cycle and plays a crucial role in gluconeogenesis and lipogenensis. A deficiency of PCX can cause lactate build up and lactic acidosis. For example, excess pyruvate can be shunted into gluconeogenesis via conversion of pyruvate into oxaloacetate, but if there is a deficiency in PCX, excess pyruvate is converted into lactate instead. Intracellular PCX expression and activity can be measured by numerous techniques including, but not limited to, RT-PCR, Northern blotting, Western Blotting, protein microarrays, or any combination thereof.

Pyruvate dehydrogenase kinase 4 (PDK4) is a kinase enzyme which inactivates pyruvate dehydrogenase by phosphorylating it using ATP. PDK4 thus participates in the regulation of the pyruvate dehydrogenase complex. Both PDK and the pyruvate dehydrogenase complex are located in the mitochondrial matrix of eukaryotes. The complex acts to convert pyruvate (a product of glycolysis in the cytosol) to acetyl-coA, which is then oxidized in the mitochondria to produce energy, in the citric acid cycle. By downregulating the activity of this complex, PDK4 will decrease the oxidation of pyruvate in the mitochondria and increase the conversion of pyruvate to lactate in the cytosol. Intracellular PDK4 expression and activity can be measured by numerous techniques including, but not limited to, RT-PCR, Northern blotting, Western Blotting, protein microarrays, or any combination thereof.

Peroxisome proliferator-activated receptor-γ coactivator (PGC)-1a is a member of a family of transcription coactivators that plays a central role in the regulation of cellular energy metabolism. PGC-1α stimulates mitochondrial biogenesis and promotes the remodeling of muscle tissue to a fiber-type composition that is metabolically more oxidative and less glycolytic in nature. It also participates in the regulation of both carbohydrate and lipid metabolism.

As further disclosed herein, Oct1 deficiency is associated with a coordinate decrease in a “sternness” gene expression signature. For example, Oct1 deficiency results in a decrease in the expression of Diap2, Stam, Gas2, Mertk, Laptm4b, Itga6, Zcchc10, Kif2a, Ndufab1, Tgs1 and Chd1.

1. Detecting Cancer Stem Cells

Thus, provided herein is a method of identifying a cancer stem cell in a biological sample, comprising assaying for the levels of Oct1 in the biological sample.

Also provided is a method of identifying a cancer stem cell in a biological sample, comprising assaying for the levels of Diap2, Stam, Gas2, Mertk, Laptm4b, Itga6, Zcchc10, Kif2a, Ndufab1, Tgs1, Chd1, or a combination thereof. Also provided is a method of identifying a cancer stem cell in a biological sample, comprising assaying for the levels of Oct1 in the biological sample and further comprising assaying for the levels of Diap2, Stam, Gas2, Mertk, Laptm4b, Itga6, Zcchc10, Kif2a, Ndufab1, Tgs1, Chd1, or a combination thereof.

In some aspects, an increase or an overexpression in the amount of Oct1 in the biological sample as compared to a negative control is an indication of the presence of cancer stem cells in the biological sample. The negative control of the disclosed method can in some aspects be a biological sample comprising cells, such as cancer cells, that do not include cancer stem cells. Thus, the negative control can be a biological sample from the subject not expected to have cancer stem cells. For example, the negative control can be surrounding tissue or tumor cells. In some aspects, the negative control is merely a reference value provided in advance. For example, the reference value can be from studies determining the average levels of Oct1 in cancer tissue that does not comprise cancer stem cells.

The disclosed method can further or alternatively comprise comparing the amount of Oct1 in the biological sample to a positive control. The positive control of the disclosed method can in some aspects be a biological sample comprising cancer stem cells. In a preferred aspect, the positive control is merely a reference value provided in advance. For example, the reference value can be from studies determining the average levels of Oct1 in cancer tissue that does comprise cancer stem cells.

Also disclosed is a method of identifying a cancer stem cell in a biological sample, comprising assaying for the levels of an Oct1 Target Protein (OTP) in the biological sample. In some aspects, the difference in the amount of the OTP in the biological sample that is overexpressing Oct1 when compared to the amount of OTP in a control is an indication of the presence of cancer stem cells in the biological sample. In some aspects, there is an overall decrease in oxidative metabolism and a decrease, increase, or a combination thereof of OTP when Oct1 is overexpressed.

In some aspects, the OTP comprises pyruvate carboxylase (PCX), pyruvate dehydrogenase kinase 4 (PDK4), alcohol dehydrogenase 1A (ALDH1a), dihyrdolipoamide acetyltransferase (Dlat), isocitrate dehydrogenase, succinate dehydrogenase, peroxisome proliferator-activated receptor-γ co-activator-1 alpha (PGC-1α), glutaminyl tRNA synthetase, glutamate-cysteine ligase, phosphoribosyl pyrophosphate amidotransferase, cabamoyl-phosphate synthetase 2, aspartate transcabamylase, dihydroorotase, glutathione reductase 1, adenylosuccinate lyase 1, or any combination thereof.

In some aspects, the OTP is pyruvate carboxylase. Thus, disclosed is a method of identifying a cancer stem cell in a biological sample, comprising assaying for the levels of pyruvate carboxylase in the biological sample. In these aspects, an increase in the amount of the pyruvate carboxylase in the biological sample when compared to the amount of pyruvate carboxylase in a control is an indication of the presence of cancer stem cells in the biological sample.

In some aspects, the OTP is alcohol dehydrogenase 1A (ALDH1a). Thus, disclosed is a method of identifying a cancer stem cell in a biological sample, comprising assaying for the levels of ALDH1a in the biological sample. In these aspects, an increase in the amount of the ALDH1a in the biological sample when compared to the amount of ALDH1a in a control is an indication of the presence of cancer stem cells in the biological sample.

In some aspects, the OTP is pyruvate dehydrogenase kinase 4 (PDK4), Dlat, isocitrate dehydrogenase, succinate dehydrogenase, peroxisome proliferator-activated receptor-γ co-activator-1 alpha (PGC-1α), or any combination thereof. Thus, in some aspects, the OTP is PDK4.

While high PDK4 can inhibit PDH and decrease oxidative metabolism, which is consistent with a stem cell phenotype, in some aspects, PDK4 expression is decreased in cancer stem cells. As disclosed herein, Oct1 represses this PDK4 and thus glucose oxidation is reduced in the (anti-tumorigenic) Oct1 KO condition. This apparent contradiction is answered by the high degree of amino acid oxidation in these cells.

Thus, in some aspects, intracellular oxidative metabolism can decrease when Oct1 is overexpressed. Further, overexpression of Oct1 can reduce PDK4 expression in cancer stem cells. Thus, in this aspect, a cancer stem cell phenotype can have an increase in Oct1 and a decrease in the amount of PDK4 when compared to a control. Thus, disclosed is a method of identifying a cancer stem cell in a biological sample, comprising assaying for the levels of PDK4 in the biological sample. In these aspects, a decrease in the amount of PDK4 in the biological sample when compared to the amount of PDK4 in a control is an indication of the presence of cancer stem cells in the biological sample.

Thus, disclosed is a method of identifying a cancer stem cell in a biological sample, comprising assaying for the levels of Dlat in the biological sample. In these aspects, a decrease in the amount of Dlat in the biological sample when compared to the amount of Dlat in a control is an indication of the presence of cancer stem cells in the biological sample.

Thus, disclosed is a method of identifying a cancer stem cell in a biological sample, comprising assaying for the levels of isocitrate dehydrogenase in the biological sample. In these aspects, a decrease in the amount of isocitrate dehydrogenase in the biological sample when compared to the amount of isocitrate dehydrogenase in a control is an indication of the presence of cancer stem cells in the biological sample.

Thus, disclosed is a method of identifying a cancer stem cell in a biological sample, comprising assaying for the levels of succinate dehydrogenase in the biological sample. In these aspects, a decrease in the amount of succinate dehydrogenase in the biological sample when compared to the amount of succinate dehydrogenase in a control is an indication of the presence of cancer stem cells in the biological sample.

Thus, disclosed is a method of identifying a cancer stem cell in a biological sample, comprising assaying for the levels of PGC-1α in the biological sample. In these aspects, a decrease in the amount of PGC-1α in the biological sample when compared to the amount of PGC-1α in a control is an indication of the presence of cancer stem cells in the biological sample.

In some aspects, Oct1, the at least one OTP, or any combination thereof, can be expressed in both cancer cells, cancer stem cells, and “normal cells.” Thus, in some aspects, the difference (i.e., an increase, decrease, or a combination thereof) in the levels Oct1, OTP, or any combination thereof, can reflect a 0.5 fold, 1 fold, 2 fold, 3 fold, 4 fold, 5 fold, 6 fold, 7 fold, 8 fold, 9 fold, 10 fold, 11 fold, 12 fold, 13 fold, 14 fold, or 15 fold difference when compared to a control (e.g., cancer or normal cells). Likewise, the difference in the levels of Oct1, OTP, or any combination thereof, can reflect an at least 1%, 25%, 50%, 75%, 100%, 150%, 200%, 250%, 300%, 350%, 400%, 450%, 500%, 600%, 650%, 700%, 750%, 800%, 850%, 900%, 950%, 1000%, 1050%, 1100%, 1150%, 1200%, 1250%, 1300%, 1350%, 1400%, 1450%, or a 1500% change when compared to a control (e.g., cancer or normal cells). For example, the levels of Oct1, ALDH1a, or any combination thereof, can be at least 0.5 fold to 15 fold, 1 fold to 12 fold, 2 fold to 10 fold, 3 fold to 8 fold, or 3 fold to 6 fold higher in cancer stem cells when compared “normal cells.”

As cancer stem cells can be rare within the cancer cells, immunohistochemical methods can be used to visualize relative increases or decreases in the expression of the OTP, Oct1, or any combination thereof within cancer stem cells. These methods can be subjective, i.e., wherein the skilled artisan identifies “positive cells” with levels above “background” levels. However, in some aspects, the methods can quantitative or semi-quantitative, e.g., by capturing the image on a computer and quantifying the pixels. Other such methods are known and contemplated herein.

In some aspects, the difference in at least two proteins selected from the group including any OTP, Oct1, or any combination thereof, can be assayed and compared to a control to determine the presence of cancer stem cells in a biological sample. In some aspects, the difference in at least three proteins selected from the group including any OTP, Oct1, or any combination thereof may be assayed and compared to a control to determine the presence of cancer stem cells in a biological sample. In some aspects, difference in at least four proteins selected from the group that including any OTP, Oct1, or any combination thereof may be assayed and compared to a control to determine the presence of cancer stem cells in a biological sample. In some aspects, difference in at least five proteins selected from the group that including any OTP, Oct1, or any combination thereof may be assayed and compared to a control to determine the presence of cancer stem cells in a biological sample.

Examples of cancers in which cancer stem cells can be detected by the disclosed methods include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. Thus, in some aspects, the cancer comprises an adenocarcinoma. For example, the adenocarcinoma can be a colon adenocarcinoma, a breast carcinoma, or a lung carcinoma.

Compositions and methods for detecting and isolating cancer stem cells in solid tumors are disclosed in U.S. Pat. No. 7,115,360, which is incorporated by reference herein in its entirety for the teaching of these methods. As disclosed therein, cancer stem cells are generally CD44⁺. Moreover, cancer stem cells are generally CD24^{-/lo}.

Thus, the herein disclosed method can further comprise assaying for the levels of CD44 in the biological sample, wherein detection of Oct1 and CD44 in the biological sample is an indication of the presence of cancer stem cells in the biological sample Likewise, the method can further comprise assaying for the levels of CD24 in the biological sample, wherein detection of Oct1 and failure to detect high levels of CD24 in the biological sample is an indication of the presence of cancer stem cells in the biological sample.

Moreover, the methods can further comprise assaying for the levels of CD2, CD3, CD10, CD14, CD16, CD31, CD45, CD64, CD140b, or a combination thereof, wherein failure to detect CD2, CD3, CD10, CD14, CD16, CD31, CD45, CD64, or CD140b in the biological sample is an indication of the presence of cancer stem cells in the biological sample. Other such combinations of biomarkers for detecting or verifying the presence of cancer stem cells in a biological sample are contemplated herein.

The biological sample of the disclosed method can in some aspects be any bodily fluid, tissue, or cells from the subject in which the assaying for the levels of cancer stem cells is desired. Thus, in some aspects, the biological sample is from a subject diagnosed with cancer. For example, the cancer can be an adenocarcinoma. Thus, the cancer can be a colon adenocarcinoma, a breast carcinoma, or a lung carcinoma. Thus, in some aspects, the biological sample comprises a tumor biopsy. However, in other aspects, the biological sample comprises blood; plasma; serum; urine; stool; spinal fluid; nipple aspirate; lymph fluid; external secretions of the skin, respiratory tract, intestinal or genitourinary tracts; bile; saliva; or milk. In some aspects, the biological sample is a constituent of in vitro cell culture of a cell from the subject. Other such sources of cancer cells are contemplated herein.

Also disclosed herein is a method of identifying altered cellular metabolism in a cell, comprising measuring Oct1 expression levels in the cell. In some aspects, the cell is a cancer stem cell. In some aspects of the method, the altered metabolism comprises an increase in glycolytic metabolism when compared to a control.

In some aspects of the method, the altered metabolism comprises a change in levels of metabolic factors. For example, the metabolic factors affected by the altered metabolism can comprise lactate, oxygen consumption, ATP production, NAD+/NADH ratio, glucose, glucose oxidation, mitochondrial membrane potential, palmitate oxidation, intracellular glutamate oxidation.

In other aspects of the method, the altered metabolism comprises a change in levels of an Oct1 Target Protein (OTP). For example, the OTP affected by the altered metabolism can comprise pyruvate carboxylase (PCX), pyruvate dehydrogenase kinase 4 (PDK4), dihyrdolipoamide acetyltransferase (Dlat), isocitrate dehydrogenase, succinate dehydrogenase, peroxisome proliferator-activated receptor-γ co-activator-1 alpha (PGC-1α), glutaminyl tRNA synthetase, glutamate-cysteine ligase, phosphoribosyl pyrophosphate amidotransferase, cabamoyl-phosphate synthetase 2, aspartate transcabamylase, dihydroorotase, glutathione reductase 1, adenylosuccinate lyase 1, or any combination thereof.

In some aspects, the altered metabolism comprises an increase in lactate, an increase in NAD+/NADH, an increase in glucose, an increase in glucose oxidation, or any combination thereof. In some aspects, the altered metabolism comprises a decrease in oxygen consumption, a decrease in ATP production, a decrease in palmitate oxidation, a decrease in glutamate oxidation, or any combination thereof.

i. Immunoassay

In some aspects, the presence of Oct1, or any of the other biomarkers disclosed herein, in the biological sample is detected using an immunoassay. Immunoassays, in their most simple and direct sense, are binding assays involving binding between antibodies and antigen. Thus, in some aspects, the method comprises detecting Oct1 using an antibody that specifically binds Oct1, such as human Oct1. Antibodies that specifically bind human Oct1 are commercially available and can be produced using routine skill

Many types and formats of immunoassays are known and all are suitable for detecting the disclosed biomarkers. Examples of immunoassays are immunohistochemistry (IHC), enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In one aspect, the immunoassay comprises immunohistochemistry, wherein individual Oct1-positive (Oct1^{high}) cells can be visualized amongst many Oct1-negative (Oct1^{low}) cells. Immunohistochemistry (IHC) refers to the process of localizing proteins in cells of a tissue section exploiting the principle of antibodies binding specifically to antigens in biological tissues. Immunohistochemical staining is widely used in the diagnosis of abnormal cells such as those found in cancerous tumors. Visualising an antibody-antigen interaction can be accomplished in a number of ways. In some aspects, an antibody is conjugated to an enzyme, such as peroxidase, that can catalyse a colour-producing reaction (see immunoperoxidase staining). In some aspects, the antibody can be tagged to a fluorophore, such as fluorescein, rhodamine, DyLight Fluor or Alexa Fluor.

In the procedure, depending on the purpose and the thickness of the experimental sample, either thin (about 4-40 μm) slices are taken of the tissue of interest, or if the tissue is not very thick and is penetrable it is used whole. The slicing is usually accomplished through the use of a microtome, and slices are mounted on slides. “Free-floating IHC” uses slices that are not mounted, these slices are normally produced using a vibrating microtome.

The tissue (e.g., tumor tissue) can be either fixed or frozen. Frozen section is a rapid way to fix and mount histology sections. It is used in surgical removal of tumors, and allows rapid determination of margin (that the tumor has been completely removed). It is done using a refrigeration device called a cryostat. The frozen tissue is sliced using a microtome, and the frozen slices are mounted on a glass slide and stained the same way as other methods. Alternatively, chemical fixatives can be used to preserve tissue from degradation, and to maintain the structure of the cells inclusive of sub-cellular components such as cell organelles (e.g., nucleus, endoplasmic reticulum, mitochondria). The most common fixative for light microscopy is 10% neutral buffered formalin (4% formaldehyde in phosphate buffered saline. These fixatives preserve tissues or cells mainly by irreversibly cross-linking proteins. The main action of these aldehyde fixatives is to cross-link amino groups in proteins through the formation of CH₂ (methylene) linkage, in the case of formaldehyde, or by a C₅H₁₀ cross-links in the case of glutaraldehyde. This process, while preserving the structural integrity of the cells and tissue can damage the biological functionality of proteins, particularly enzymes, and can also denature them to a certain extent.

Biological tissue must be supported in a hard matrix to allow sufficiently thin sections to be cut, typically 5 μm (micrometres; 1000 micrometres=1 mm) thick for light microscopy. For light microscopy, paraffin wax is most frequently used. Since it is immiscible with water, the main constituent of biological tissue, water must first be removed in the process of dehydration. Samples can be transferred through baths of progressively more concentrated ethanol to remove the water, followed by a clearing agent, usually xylene, to remove the alcohol, and finally molten paraffin wax which replaces the xylene.

After the tissues have been dehydrated and infiltrated with the embedding material they are ready for embedding. During this process the tissue samples are placed into moulds along with liquid embedding material which is then hardened. This is achieved by cooling in the case of paraffin wax.

Embedding can also be accomplished using frozen, non-fixed tissue in a water-based medium. Pre-frozen tissues are placed into moulds with the liquid embedding material, usually a water-based glycol or resin, which is then frozen to form hardened blocks.

There are two strategies used for the immunohistochemical detection of antigens in tissue, the direct method and the indirect method. In both cases, many antigens also need an additional step for unmasking, which often makes the difference between staining and no staining. Unlike immunocytochemistry, the tissue does not need to be permeabilized because this has already been accomplished by the microtome blade during sample preparation. Detergents like Triton X-100 are generally used in immunohistochemistry to reduce surface tension, allowing less reagent to be used to achieve better and more even coverage of the sample.

The direct method is a one-step staining method, and involves a labeled antibody (e.g. FITC conjugated antiserum) reacting directly with the antigen in tissue sections. This technique utilizes only one antibody and the procedure is therefore simple and rapid. However, it can suffer problems with sensitivity due to little signal amplification and is in less common use than indirect methods.

The indirect method involves an unlabeled primary antibody (first layer) which reacts with tissue antigen, and a labeled secondary antibody (second layer) which reacts with the primary antibody. (The secondary antibody must be raised against the IgG of the animal species in which the primary antibody has been raised.) This method is more sensitive due to signal amplification through several secondary antibody reactions with different antigenic sites on the primary antibody. The second layer antibody can be labeled with a fluorescent dye or an enzyme. The indirect method, aside from its greater sensitivity, also has the advantage that only a relatively small number of standard conjugated (labeled) secondary antibodies needs to be generated.

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label. See, for example, U.S. Pat. Nos. 3,817,837; 3,850,752; 3,939,350; 3,996,345; 4,277,437; 4,275,149 and 4,366,241, each of which is incorporated herein by reference in its entirety and specifically for teachings regarding immunodetection methods and labels.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

A modifier unit such as a radionuclide can be incorporated into or attached directly to any of the compounds described herein by halogenation. In another aspect, the radionuclide can be attached to a linking group or bound by a chelating group, which is then attached to the compound directly or by means of a linker. Radiolabeling techniques such as these are routinely used in the radiopharmaceutical industry.

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-genrating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

ii. Nucleic Acid Detection

In some aspects, the method comprises detecting Oct1, or any of the other biomarkers disclosed herein, using a primer or probe that selectively binds Oct1 mRNA.

A number of widely used procedures exist for detecting and determining the abundance of a particular mRNA in a total or poly(A) RNA sample. For example, specific mRNAs can be detected using Northern blot analysis, nuclease protection assays (NPA), in situ hybridization, or reverse transcription-polymerase chain reaction (RT-PCR).

In theory, each of these techniques can be used to detect specific RNAs and to precisely determine their expression level. In general, Northern analysis is the only method that provides information about transcript size, whereas NPAs are the easiest way to simultaneously examine multiple messages. In situ hybridization is used to localize expression of a particular gene within a tissue or cell type, and RT-PCR is the most sensitive method for detecting and quantitating gene expression.

Northern analysis presents several advantages over the other techniques. The most compelling of these is that it is the easiest method for determining transcript size, and for identifying alternatively spliced transcripts and multigene family members. It can also be used to directly compare the relative abundance of a given message between all the samples on a blot. The Northern blotting procedure is straightforward and provides opportunities to evaluate progress at various points (e.g., intactness of the RNA sample and how efficiently it has transferred to the membrane). RNA samples are first separated by size via electrophoresis in an agarose gel under denaturing conditions. The RNA is then transferred to a membrane, crosslinked and hybridized with a labeled probe. Nonisotopic or high specific activity radiolabeled probes can be used including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes.

The Nuclease Protection Assay (NPA) (including both ribonuclease protection assays and S1 nuclease assays) is an extremely sensitive method for the detection and quantitation of specific mRNAs. The basis of the NPA is solution hybridization of an antisense probe (radiolabeled or nonisotopic) to an RNA sample. After hybridization, single-stranded, unhybridized probe and RNA are degraded by nucleases. The remaining protected fragments are separated on an acrylamide gel. Solution hybridization is typically more efficient than membrane-based hybridization, and it can accommodate up to 100 μg of sample RNA, compared with the 20-30 μg maximum of blot hybridizations. NPAs are also less sensitive to RNA sample degradation than Northern analysis since cleavage is only detected in the region of overlap with the probe (probes are usually about 100-400 bases in length).

NPAs are the method of choice for the simultaneous detection of several RNA species. During solution hybridization and subsequent analysis, individual probe/target interactions are completely independent of one another. Thus, several RNA targets and appropriate controls can be assayed simultaneously (up to twelve have been used in the same reaction), provided that the individual probes are of different lengths. NPAs are also commonly used to precisely map mRNA termini and intron/exon junctions.

In situ hybridization (ISH) is a powerful and versatile tool for the localization of specific mRNAs in cells or tissues. Unlike Northern analysis and nuclease protection assays, ISH does not require the isolation or electrophoretic separation of RNA. Hybridization of the probe takes place within the cell or tissue. Since cellular structure is maintained throughout the procedure, ISH provides information about the location of mRNA within the tissue sample.

The procedure begins by fixing samples in neutral-buffered formalin, and embedding the tissue in paraffin. The samples are then sliced into thin sections and mounted onto microscope slides. (Alternatively, tissue can be sectioned frozen and post-fixed in paraformaldehyde.) After a series of washes to dewax and rehydrate the sections, a Proteinase K digestion is performed to increase probe accessibility, and a labeled probe is then hybridized to the sample sections. Radiolabeled probes are visualized with liquid film dried onto the slides, while nonisotopically labeled probes are conveniently detected with colorimetric or fluorescent reagents.

RT-PCR has revolutionized the study of gene expression. It is now theoretically possible to detect the RNA transcript of any gene, regardless of the scarcity of the starting material or relative abundance of the specific mRNA. In RT-PCR, an RNA template is copied into a complementary DNA (cDNA) using a retroviral reverse transcriptase. The cDNA is then amplified exponentially by PCR. As with NPAs, RT-PCR is somewhat tolerant of degraded RNA. As long as the RNA is intact within the region spanned by the primers, the target will be amplified.

Relative quantitative RT-PCR involves amplifying an internal control simultaneously with the gene of interest. The internal control is used to normalize the samples. Once normalized, direct comparisons of relative abundance of a specific mRNA can be made across the samples. It is crucial to choose an internal control with a constant level of expression across all experimental samples (i.e., not affected by experimental treatment). Commonly used internal controls (e.g., GAPDH, β-actin, cyclophilin) often vary in expression and, therefore, may not be appropriate internal controls. Additionally, most common internal controls are expressed at much higher levels than the mRNA being studied. For relative RT-PCR results to be meaningful, all products of the PCR reaction must be analyzed in the linear range of amplification. This becomes difficult for transcripts of widely different levels of abundance.

Competitive RT-PCR is used for absolute quantitation. This technique involves designing, synthesizing, and accurately quantitating a competitor RNA that can be distinguished from the endogenous target by a small difference in size or sequence. Known amounts of the competitor RNA are added to experimental samples and RT-PCR is performed. Signals from the endogenous target are compared with signals from the competitor to determine the amount of target present in the sample.

### 2. Treating Cancer

Also disclosed herein is a method of treating cancer stem cells in a subject, comprising administering to the subject an inhibitor of Oct1 activity. “Activities” of a protein include, for example, transcription, translation, intracellular translocation, secretion, phosphorylation by kinases, cleavage by proteases, homophilic and heterophilic binding to other proteins, ubiquitination. Notably, as disclosed herein, Oct1 acts as a transcription factor. Thus, the inhibitor of the disclosed methods can be any nucleic acid, peptide, protein, molecule, or compound that inhibits one or more of the activities of Oct1 known or shown to be necessary for its activity as a transcription factor.

In some aspects of the method, the subject has been diagnosed as having cancer stem cells. For example, in some aspects, the subject has been diagnosed with cancer cells expressing Oct1, CD44, or a combination thereof. In some aspects of the method, the subject has undergone or been prescribed irradiation, chemotherapy, or a combination thereof.

In some aspects, the method further comprises administering to the subject a modulator of an Oct1 Target Protein (OTP). In some aspects, the OTP is pyruvate carboxylase (PCX). Thus, in some aspects, the method further comprises administering to the subject an inhibitor of PCX.

In some aspects, the OTP is pyruvate dehydrogenase kinase 4 (PDK4), dihyrdolipoamide acetyltransferase (Dlat), isocitrate dehydrogenase, succinate dehydrogenase, peroxisome proliferator-activated receptor-γ co-activator-1 alpha (PGC-1α), or any combination thereof. Thus, in some aspects, the method further comprises administering to the subject an agonist of PDK4, Dlat, isocitrate dehydrogenase, succinate dehydrogenase, PGC-1α, or any combination thereof.

i. Functional Nucleic Acids

In some aspects, the inhibitor of the disclosed methods, such as the Oct1 inhibitor, is a functional nucleic acid. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, RNAi, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of Oct1 or the genomic DNA of Oct1 or they can interact with the polypeptide Oct1. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹². A representative sample of methods and techniques which aid in the design and use of antisense molecules can be found in U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with K_{d}'s from the target molecule of less than 10-12 M. It is preferred that the aptamers bind the target molecule with a K_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule (U.S. Pat. No. 5,543,293). It is preferred that the aptamer have a K_{d} with the target molecule at least 10, 100, 1000, 10,000, or 100,000 fold lower than the K_{d} with a background binding molecule. It is preferred when doing the comparison for a polypeptide for example, that the background molecule be a different polypeptide. Representative examples of how to make and use aptamers to bind a variety of different target molecules can be found in U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203; International Patent Application Nos. WO 9858058 by Ludwig and Sproat, WO 9858057 by Ludwig and Sproat, and WO 9718312 by Ludwig and Sproat) hairpin ribozymes (for example, U.S. Pat. Nos. 5,631,115, 5,646,031, 5,683,902, 5,712,384, 5,856,188, 5,866,701, 5,869,339, and 6,022,962), and tetrahymena ribozymes (for example, U.S. Pat. Nos. 5,595,873 and 5,652,107). There are also a number of ribozymes that are not found in natural systems, but which have been engineered to catalyze specific reactions de novo (for example, U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹². Representative examples of how to make and use triplex forming molecules to bind a variety of different target molecules can be found in U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in U.S. Pat. Nos. 5,168,053, 5,624,824, 5,683,873, 5,728,521, 5,869,248, and 5,877,162.

Gene expression can also be effectively silenced in a highly specific manner through RNA interference (RNAi). This silencing was originally observed with the addition of double stranded RNA (dsRNA) (Fire, A., et al. (1998) Nature, 391:806-11; Napoli, C., et al. (1990) Plant Cell 2:279-89; Hannon, G. J. (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, S. M., et al. (2001) Genes Dev., 15:188-200; Bernstein, E., et al. (2001) Nature, 409:363-6; Hammond, S. M., et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, A., et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, J., et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al. (2001) Nature, 411:494 498) (Ui-Tei, K., et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit. Disclosed herein are any siRNA designed as described above based on the sequences for Oct1.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors. Disclosed herein are any shRNA designed as described above based on the sequences for Oct1

MicroRNAs (miRNA or μRNA) are single-stranded RNA molecules of 21-23 nucleotides in length, which regulate gene expression. miRNAs are encoded by genes from whose DNA they are transcribed but miRNAs are not translated into protein (i.e. they are non-coding RNAs); instead each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression. Disclosed herein are any miRNA designed as described above based on the sequences for Oct1.

As shown in the example data below, it may be advantageous to either reduce or inhibit Oct1 expression in cells overexpressing Oct1. Cells that overexpress Oct1 can include, but are not limited to, cancer cells and cancer stem cells. In one aspect, Oct1 expression can be reduced or inhibited by administering a functional nucleic acid, such as an siRNA that hybridizes to an Oct1 mRNA transcript, an miRNA that hybridizes to an Oct1 mRNA transcript, a shRNA that encodes for an siRNA or miRNA that hybridizes to an Oct1 mRNA transcript, or a combination thereof to a cell that overexpresses Oct1 or a molecular variant thereof. In this aspect, intracellular gene silencing can be initiated and Oct1 expression can be decreased. In one aspect, the siRNA or the shRNA encoding the siRNA has the following nucleotide sequence 5′GCCTTGAACCTCAGCTTTAAG3′ (SEQ ID NO: 2).

ii. Pharmaceutical Compositions

The Oct1 inhibitor disclosed herein can be used therapeutically in combination with a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

iii. Therapeutic Administration

The herein disclosed Oct1 inhibitors, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained. See, e.g., U.S. Pat. No. 3,610,795, which is incorporated by reference herein.

The compositions disclosed herein may be administered prophylactically to patients or subjects who are at risk for colorectal cancer. Thus, the method can further comprise identifying a subject at risk metastatic colorectal cancer prior to administration of the herein disclosed Oct1 inhibitors.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

iv. Combination Therapies

Provided herein is a composition that comprises a Oct1 inhibitor and any known or newly discovered substance that can be administered to the site of a cancer.

Numerous anti-cancer (antineoplastic) drugs are available for combination with the present method and compositions. Antineoplastic drugs include Acivicin, Aclarubicin, Acodazole Hydrochloride, AcrQnine, Adozelesin, Aldesleukin, Altretamine, Ambomycin, Ametantrone Acetate, Aminoglutethimide, Amsacrine, Anastrozole, Anthramycin, Asparaginase, Asperlin, Azacitidine, Azetepa, Azotomycin, Batimastat, Benzodepa, Bicalutamide, Bisantrene Hydrochloride, Bisnafide Dimesylate, Bizelesin, Bleomycin Sulfate, Brequinar Sodium, Bropirimine, Busulfan, Cactinomycin, Calusterone, Caracemide, Carbetimer, Carboplatin, Carmustine, Carubicin Hydrochloride, Carzelesin, Cedefingol, Chlorambucil, Cirolemycin, Cisplatin, Cladribine, Crisnatol Mesylate, Cyclophosphamide, Cytarabine, Dacarbazine, Dactinomycin, Daunorubicin Hydrochloride, Decitabine, Dexormaplatin, Dezaguanine, Dezaguanine Mesylate, Diaziquone, Docetaxel, Doxorubicin, Doxorubicin Hydrochloride, Droloxifene, Droloxifene Citrate, Dromostanolone Propionate, Duazomycin, Edatrexate, Eflomithine Hydrochloride, Elsamitrucin, Enloplatin, Enpromate, Epipropidine, Epirubicin Hydrochloride, Erbulozole, Esorubicin Hydrochloride, Estramustine, Estramustine Phosphate Sodium, Etanidazole, Ethiodized Oil I 131, Etoposide, Etoposide Phosphate, Etoprine, Fadrozole Hydrochloride, Fazarabine, Fenretinide, Floxuridine, Fludarabine Phosphate, Fluorouracil, Fluorocitabine, Fosquidone, Fostriecin Sodium, Gemcitabine, Gemcitabine Hydrochloride, Gold Au 198, Hydroxyurea, Idarubicin Hydrochloride, Ifosfamide, Ilmofosine, Interferon Alfa-2a, Interferon Alfa-2b, Interferon Alfa-n1, Interferon Alfa-n3, Interferon Beta-I a, Interferon Gamma-Ib, Iproplatin, Irinotecan Hydrochloride, Lanreotide Acetate, Letrozole, Leuprolide Acetate, Liarozole Hydrochloride, Lometrexol Sodium, Lomustine, Losoxantrone Hydrochloride, Masoprocol, Maytansine, Mechlorethamine Hydrochloride, Megestrol Acetate, Melengestrol Acetate, Melphalan, Menogaril, Mercaptopurine, Methotrexate, Methotrexate Sodium, Metoprine, Meturedepa, Mitindomide, Mitocarcin, Mitocromin, Mitogillin, Mitomalcin, Mitomycin, Mitosper, Mitotane, Mitoxantrone Hydrochloride, Mycophenolic Acid, Nocodazole, Nogalamycin, Ormaplatin, Oxisuran, Paclitaxel, Pegaspargase, Peliomycin, Pentamustine, Peplomycin Sulfate, Perfosfamide, Pipobroman, Piposulfan, Piroxantrone Hydrochloride, Plicamycin, Plomestane, Porfimer Sodium, Porfiromycin, Prednimustine, Procarbazine Hydrochloride, Puromycin, Puromycin Hydrochloride, Pyrazofurin, Riboprine, Rogletimide, Safmgol, Safingol Hydrochloride, Semustine, Simtrazene, Sparfosate Sodium, Sparsomycin, Spirogermanium Hydrochloride, Spiromustine, Spiroplatin, Streptonigrin, Streptozocin, Strontium Chloride Sr 89, Sulofenur, Talisomycin, Taxane, Taxoid, Tecogalan Sodium, Tegafur, Teloxantrone Hydrochloride, Temoporfin, Teniposide, Teroxirone, Testolactone, Thiamiprine, Thioguanine, Thiotepa, Tiazofurin, Tirapazamine, Topotecan Hydrochloride, Toremifene Citrate, Trestolone Acetate, Triciribine Phosphate, Trimetrexate, Trimetrexate Glucuronate, Triptorelin, Tubulozole Hydrochloride, Uracil Mustard, Uredepa, Vapreotide, Verteporfin, Vinblastine Sulfate, Vincristine Sulfate, Vindesine, Vindesine Sulfate, Vinepidine Sulfate, Vinglycinate Sulfate, Vinleurosine Sulfate, Vinorelbine Tartrate, Vinrosidine Sulfate, Vinzolidine Sulfate, Vorozole, Zeniplatin, Zinostatin, Zorubicin Hydrochloride.

Other anti-neoplastic compounds include: 20-epi-1,25 dihydroxyvitamin D3, 5-ethynyluracil, abiraterone, aclarubicin, acylfulvene, adecypenol, adozelesin, aldesleukin, ALL-TK antagonists, altretamine, ambamustine, amidox, amifostine, aminol evulinic acid, amrubicin, atrsacrine, anagrelide, anastrozole, andrographolide, angiogenesis inhibitors, antagonist D, antagonist G, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthionine sulfoximine, calcipotriol, calphostin C, camptothecin derivatives, canarypox IL-2, capecitabine, carboxamide-amino-triazole, carboxyamidotriazole, CaRest M3, CARN 700, cartilage derived inhibitor, carzelesin, casein kinase inhibitors (ICOS), castanospermine, cecropin B, cetrorelix, chlorines, chloroquinoxaline sulfonamide, cicaprost, cis-porphyrin, cladribine, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexifosfamide, dexrazoxane, dexverapamil, diaziquone, didemnin B, didox, diethylnorspermine, dihydro-5-azacytidine, dihydrotaxol, 9-dioxamycin, diphenyl spiromustine, docosanol, dolasetron, doxifluridine, droloxifene, dronabinol, duocannycin SA, ebselen, ecomustine, edelfosine, edrecolomab, eflornithine, elemene, emitefur, epirubicin, epristeride, estramustine analogue, estrogen agonists, estrogen antagonists, etanidazole, etoposide phosphate, exemestane, fadrozole, fazarabine, fenretinide, filgrastim, fmasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifene, idramantone, ilmofosine, ilomastat, imidazoacridones, imiquimod, immunostimulant peptides, insulin-like growth factor-1 receptor inhibitor, interferon agonists, interferons, interleukins, iobenguane, iododoxorubicin, ipomeanol, 4-irinotecan, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mismatched double stranded RNA, mitoguazone, mitolactol, mitomycin analogues, mitonafide, mitotoxin fibroblast growth factor-saporin, mitoxantrone, mofarotene, molgramostim, monoclonal antibody, human chorionic gonadotrophin, monophosphoryl lipid A+myobacterium cell wall sk, mopidamol, multiple drug resistance genie inhibitor, multiple tumor suppressor 1-based therapy, mustard anticancer agent, mycaperoxide B, mycobacterial cell wall extract, myriaporone, N-acetyldinaline, N-substituted benzamides, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, O6-benzylguanine, octreotide, okicenone, oligonucleotides, onapristone, ondansetron, ondansetron, oracin, oral cytokine inducer, ormaplatin, osaterone, oxaliplatin, oxaunomycin, paclitaxel analogues, paclitaxel derivatives, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, propyl bis-acridone, prostaglandin J2, proteasome inhibitors, protein A-based immune modulator, protein kinase C inhibitor, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylene conjugate, raf antagonists, raltitrexed, ramosetron, ras farnesyl protein transferase inhibitors, ras inhibitors, ras-GAP inhibitor, retelliptine demethylated, rhenium Re 186 etidronate, rhizoxin, ribozymes, RII retinamide, rogletimide, rohitukine, romurtide, roquinimex, rubiginone B1, ruboxyl, safingol, saintopin, SarCNU, sarcophytol A, sargramostim, Sdi 1 mimetics, semustine, senescence derived inhibitor 1, sense oligonucleotides, signal transduction inhibitors, signal transduction modulators, single chain antigen binding protein, sizofuran, sobuzoxane, sodium borocaptate, sodium phenylacetate, solverol, somatomedin binding protein, sonermin, sparfosic acid, spicamycin D, spiromustine, splenopentin, spongistatin 1, squalamine, stem cell inhibitor, stem-cell division inhibitors, stipiamide, stromelysin inhibitors, sulfmosine, superactive vasoactive intestinal peptide antagonist, suradista, suramin, swainsonine, synthetic glycosaminoglycans, tallimustine, tamoxifen methiodide, tauromustine, tazarotene, tecogalan sodium, tegafur, tellurapyrylium, telomerase inhibitors, temoporfin, temozolomide, teniposide, tetrachlorodecaoxide, tetrazomine, thaliblastine, thalidomide, thiocoraline, thrombopoietin, thrombopoietin mimetic, thymalfasin, thymopoietin receptor agonist, thymotrinan, thyroid stimulating hormone, tin ethyl etiopurpurin, tirapazamine, titanocene dichloride, topotecan, topsentin, toremifene, totipotent stem cell factor, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factor, urokinase receptor antagonists, vapreotide, variolin B, vector system, erythrocyte gene therapy, velaresol, veramine, verdins, verteporfin, vinorelbine, vinxaltine, vitaxin, vorozole, zanoterone, zeniplatin, zilascorb, zinostatin stimalamer.

The herein provide composition can further comprise one or more additional radiosensitizers. Examples of known radiosensitizers include gemcitabine, 5-fluorouracil, pentoxifylline, and vinorelbine. (Zhang et al., 1998; Lawrence et al., 2001; Robinson and Shewach, 2001; Strunz et al., 2002; Collis et al., 2003; Zhang et al., 2004).

In other aspects, the provided composition(s) can further comprise one or more of classes of antibiotics (e.g., Aminoglycosides, Cephalosporins, Chloramphenicol, Clindamycin, Erythromycins, Fluoroquinolones, Macrolides, Azolides, Metronidazole, Penicillins, Tetracyclines, Trimethoprim-sulfamethoxazole, Vancomycin), steroids (e.g., Andranes (e.g., Testosterone), Cholestanes (e.g., Cholesterol), Cholic acids (e.g., Cholic acid), Corticosteroids (e.g., Dexamethasone), Estraenes (e.g., Estradiol), Pregnanes (e.g., Progesterone), narcotic and non-narcotic analgesics (e.g., Morphine, Codeine, Heroin, Hydromorphone, Levorphanol, Meperidine, Methadone, Oxydone, Propoxyphene, Fentanyl, Methadone, Naloxone, Buprenorphine, Butorphanol, Nalbuphine, Pentazocine), anti-inflammatory agents (e.g., Alclofenac, Alclometasone Dipropionate, Algestone Acetonide, alpha Amylase, Amcinafal, Amcinafide, Amfenac Sodium, Amiprilose Hydrochloride, Anakinra, Anirolac, Anitrazafen, Apazone, Balsalazide Disodium, Bendazac, Benoxaprofen, Benzydamine Hydrochloride, Bromelains, Broperamole, Budesonide, Carprofen, Cicloprofen, Cintazone, Cliprofen, Clobetasol Propionate, Clobetasone Butyrate, Clopirac, Cloticasone Propionate, Cormethasone Acetate, Cortodoxone, Decanoate, Deflazacort, Delatestryl, Depo-Testosterone, Desonide, Desoximetasone, Dexamethasone Dipropionate, Diclofenac Potassium, Diclofenac Sodium, Diflorasone Diacetate, Diflumidone Sodium, Diflunisal, Difluprednate, Diftalone, Dimethyl Sulfoxide, Drocinonide, Endrysone, Enlimomab, Enolicam Sodium, Epirizole, Etodolac, Etofenamate, Felbinac, Fenamole, Fenbufen, Fenclofenac, Fenclorac, Fendosal, Fenpipalone, Fentiazac, Flazalone, Fluazacort, Flufenamic Acid, Flumizole, Flunisolide Acetate, Flunixin, Flunixin Meglumine, Fluocortin Butyl, Fluorometholone Acetate, Fluquazone, Flurbiprofen, Fluretofen, Fluticasone Propionate, Furaprofen, Furobufen, Halcinonide, Halobetasol Propionate, Halopredone Acetate, Ibufenac, Ibuprofen, Ibuprofen Aluminum, Ibuprofen Piconol, Ilonidap, Indomethacin, Indomethacin Sodium, Indoprofen, Indoxole, Intrazole, Isoflupredone Acetate, Isoxepac, Isoxicam, Ketoprofen, Lofemizole Hydrochloride, Lomoxicam, Loteprednol Etabonate, Meclofenamate Sodium, Meclofenamic Acid, Meclorisone Dibutyrate, Mefenamic Acid, Mesalamine, Meseclazone, Mesterolone, Methandrostenolone, Methenolone, Methenolone Acetate, Methylprednisolone Suleptanate, Morniflumate, Nabumetone, Nandrolone, Naproxen, Naproxen Sodium, Naproxol, Nimazone, Olsalazine Sodium, Orgotein, Orpanoxin, Oxandrolane, Oxaprozin, Oxyphenbutazone, Oxymetholone, Paranyline Hydrochloride, Pentosan Polysulfate Sodium, Phenbutazone Sodium Glycerate, Pirfenidone, Piroxicam, Piroxicam Cinnamate, Piroxicam Olamine, Pirprofen, Prednazate, Prifelone, Prodolic Acid, Proquazone, Proxazole, Proxazole Citrate, Rimexolone, Romazarit, Salcolex, Salnacedin, Salsalate, Sanguinarium Chloride, Seclazone, Sermetacin, Stanozolol, Sudoxicam, Sulindac, Suprofen, Talmetacin, Talniflumate, Talosalate, Tebufelone, Tenidap, Tenidap Sodium, Tenoxicam, Tesicam, Tesimide, Testosterone, Testosterone Blends, Tetrydamine, Tiopinac, Tixocortol Pivalate, Tolmetin, Tolmetin Sodium, Triclonide, Triflumidate, Zidometacin, Zomepirac Sodium), or anti-histaminic agents (e.g., Ethanolamines (like diphenhydrmine carbinoxamine), Ethylenediamine (like tripelennamine pyrilamine), Alkylamine (like chlorpheniramine, dexchlorpheniramine, brompheniramine, triprolidine), other anti-histamines like astemizole, loratadine, fexofenadine, Bropheniramine, Clemastine, Acetaminophen, Pseudoephedrine, Triprolidine).

### 3. Screening for Anti-Cancer Drugs

Also disclosed herein is a method of identifying an agent for use in treating a cancer stem cell, comprising contacting a sample comprising Oct1 with a candidate agent and assaying for Oct1 activity in the sample. In some aspects of the method, a decrease in Oct1 activity in the sample is an indication that the candidate agent is an effective agent for use in treating cancer stem cells. In certain aspects, the candidate agent either modulates Oct1 by decreasing its activity, or in other aspects, the candidate agent substantially or completely inhibits Oct1 activity.

The sample comprising Oct1 can be a cell over expressing Oct1, which can be termed Oct1^{Hi}. In some aspects the cell is a cancer stem cell. For example, the cancer stem cell can be from an adenocarcinoma. In other aspects, the cell is from cell line that naturally expresses Oct1. Likewise, the cell can be from a cell line that recombinantly expresses Oct1.

In some aspects, the method comprises detecting levels of Oct1 in the sample. Thus, in some aspects, a decrease in the levels of Oct1 is an indication that the candidate agent is an effective agent for use in treating cancer stem cells.

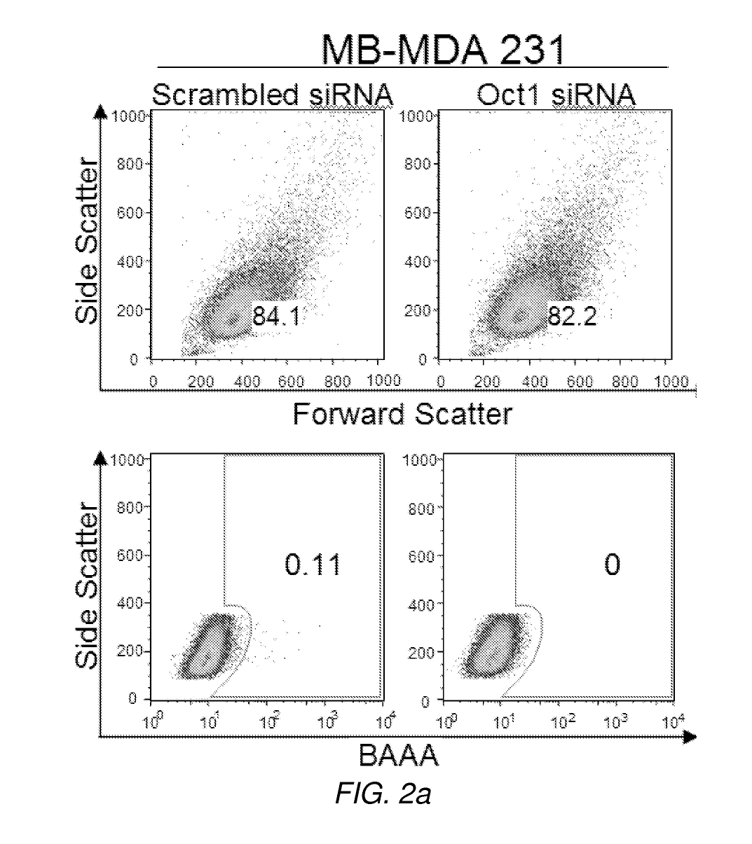

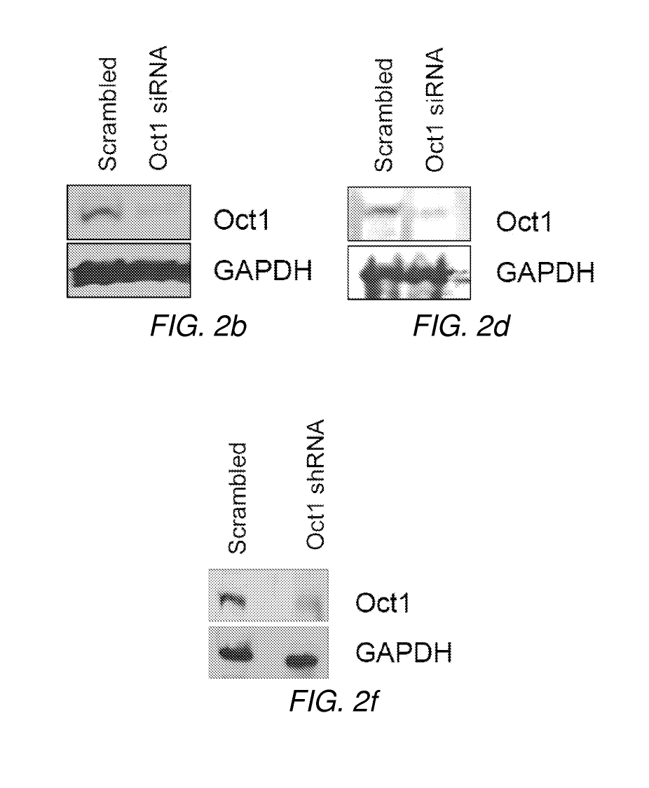

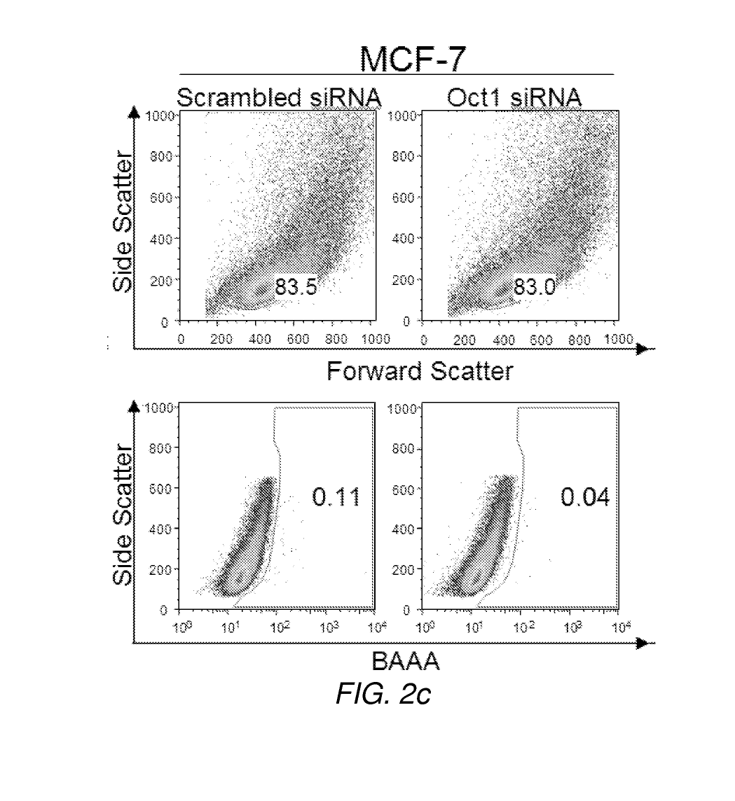

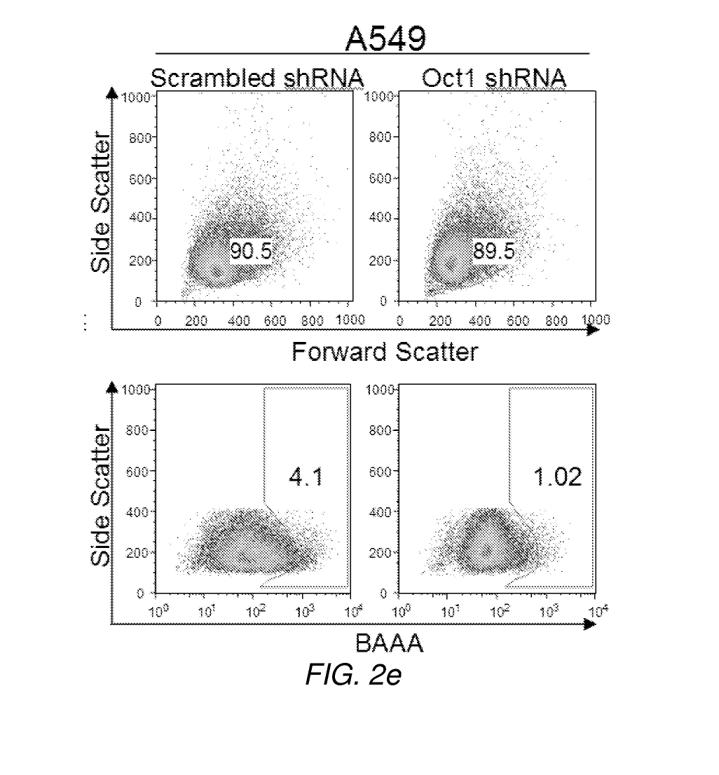

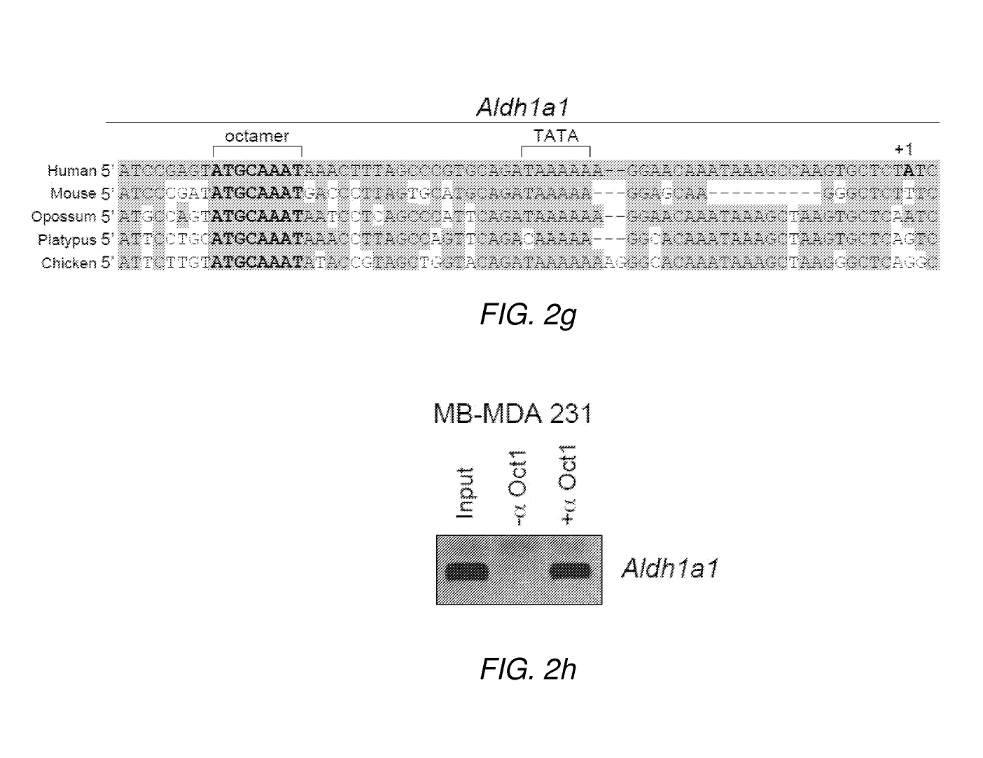

As disclosed herein, Oct1 can act as a transcription factor. For example, a description of the general mechanism for transcription regulation by Oct1 in response to genotoxic and oxidative stress is described in Kang, J., et al. (Genes Dev. 2009 Jan. 15; 23(2):208-22), which is incorporated by reference herein for the teaching of this mechanism. Likewise, FIG. 2b shows a conserved Oct1-binding site in the immediate promoter region of the Aldh1a1 promoter in several example vertebrate species. Based on this and other knowledge available in the art, the skilled artisan can design candidate inhibitors of Oct1 to, for example, prevent the binding of the Oct1 DNA binding domain to its cognate DNA sequence, or inhibit Oct1 phosphorylation. In some aspects, the candidate inhibitors can affect the on/off binding rates of Oct1 to its cognate DNA sequence. In some aspects, these candidate inhibitors selectively bind to regions of Oct1 that affect the binding of Oct1 to a subset of target DNA(s) but do not affect the binding of Oct1 to other target DNA(s). In these aspects, the candidate inhibitor can be more selective and thereby have fewer unnecessary side effects.

Thus, in some aspects, the method comprises assaying for the binding of Oct1 to its DNA binding domain. In other aspects, the method comprises assaying for transcription activation of its target gene.

Thus, the method can comprise providing a sample comprising Oct1 under conditions that allow the binding of Oct1 and, for example, Aldh1a1 promoter, contacting the sample with a candidate agent, detecting the level of Oct1/Aldh1a1 promoter binding, comparing the binding level to a control, a decrease in Oct1/Aldh1a1 promoter binding compared to the control identifying an agent that can be used to treat an inflammatory disease.

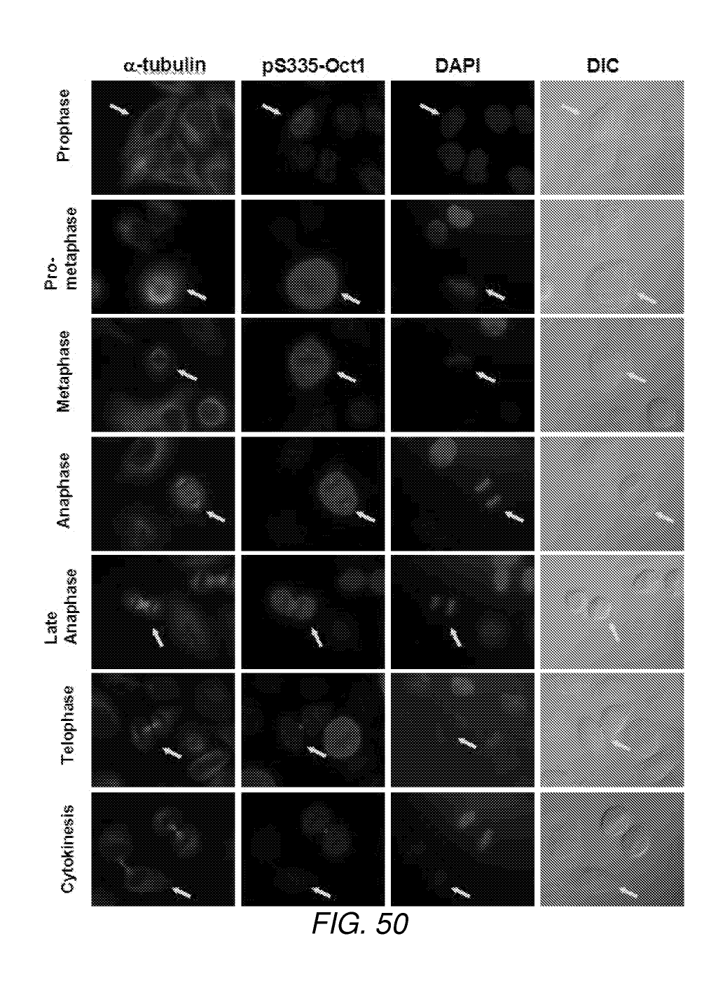

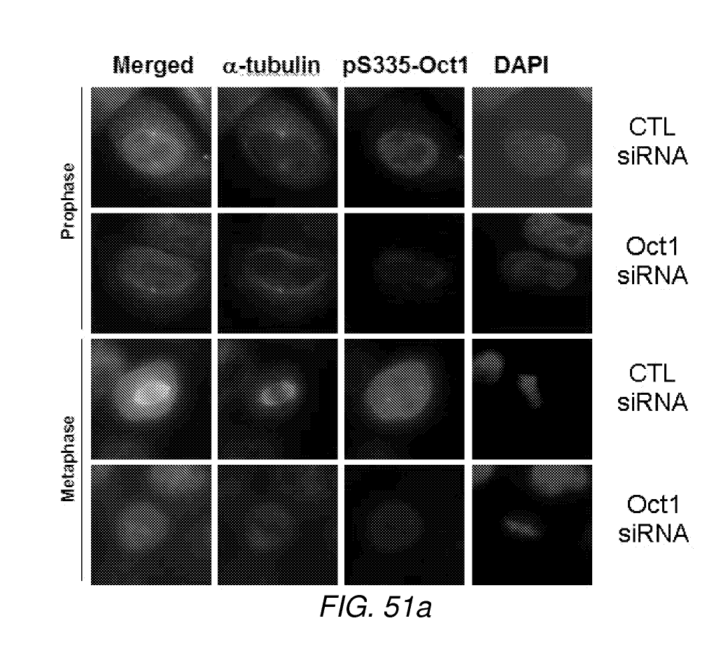

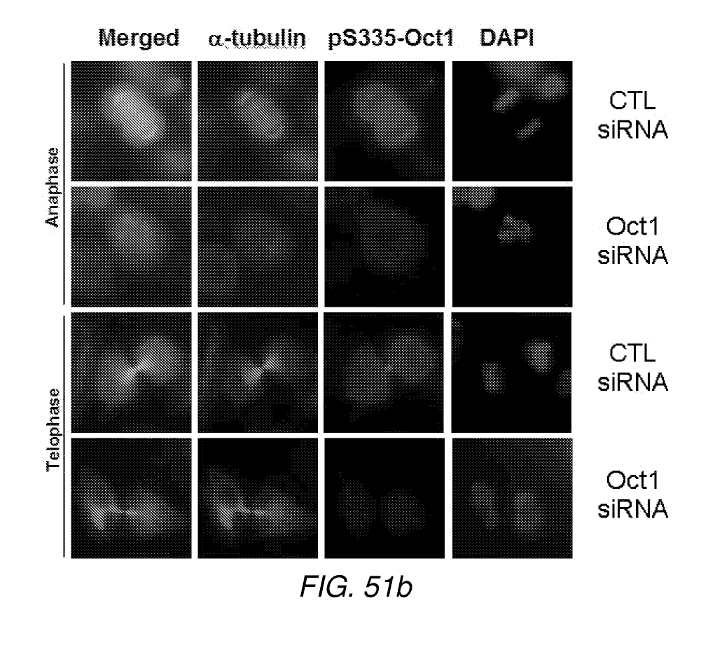

In some aspects, Oct1 can regulate asymmetric cell division in stem cells through non-transcriptional means. As disclosed herein, the form of phosphorylated Oct1 that regulates mitosis does not bind DNA (FIG. 50). Oct1 loss-of-function in cells also lacking p53 function, but not those with functional p53, leads to abnormal mitoses, indicating a redundant role for Oct1 and p53 in regulating mitotic events and indicating that targeting Oct1 can be particularly useful in malignancies lacking p53 function. Phosphorylated Oct1 interacts with the centrioles and spindle pole bodies, and it is known that stem cells asymmetrically segregate mother and daughter centrioles. Given the role of Oct1 in regulating stem cell identity, it is likely that Oct1 also regulates asymmetric cell division in a non-transcriptional context. Thus, also disclosed herein is a method of identifing candidate therapeutics using metrics of asymmetric cell division, such as loss of stem cell potential in vivo and direct microscopic visualization in a model system developed in culture.

Also disclosed herein is a method of identifying an agent for use in treating a cancer stem cell, comprising contacting a sample comprising an Oct1 Target Protein (OTP) with a candidate agent and assaying for OTP activity in the sample.

In some aspects, the OTP comprises pyruvate carboxylase (PCX). Thus, in some aspects of the method, a decrease in PCX activity in the sample is an indication that the candidate agent is an effective agent for use in treating cancer stem cells.

In some aspects, the OTP comprises pyruvate dehydrogenase kinase 4 (PDK4), dihyrdolipoamide acetyltransferase (Dlat), isocitrate dehydrogenase, succinate dehydrogenase, peroxisome proliferator-activated receptor-γ co-activator-1 alpha (PGC-1α), or any combination thereof. Thus, in some aspects of the method, an increase in PDK4, Dlat, isocitrate dehydrogenase, succinate dehydrogenase, or PGC-1α activity in the sample is an indication that the candidate agent is an effective agent for use in treating cancer stem cells.

In some aspects, the OTP comprises glutaminyl tRNA synthetase, glutamate-cysteine ligase, phosphoribosyl pyrophosphate amidotransferase, cabamoyl-phosphate synthetase 2, aspartate transcabamylase, dihydroorotase, glutathione reductase 1, adenylosuccinate lyase 1, or any combination thereof.

The levels of Oct1 in the sample or its binding to target molecules can be detected using routine methods, such as immunodetection methods, e.g., those that do not disturb protein binding. The methods can be cell-based or cell-free assays.

In some aspects, the method comprises the use of small-molecule microarrays (SMMs). See e.g. Vegas A J, Fuller J H, Koehler A N. Small-molecule microarrays as tools in ligand discovery. Chem Soc Rev. 2008 July; 37(7):1385-94. Generally, simple and general binding assays involving small-molecule microarrays can be used to identify probes for nearly any protein in the proteome. The assay can be used to identify ligands for proteins in the absence of knowledge about structure or function.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods. In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their effect on the activity of Oct1 should be employed whenever possible.

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that stimulates or inhibits Oct1. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions, such as those disclosed herein.

Candidate agents encompass numerous chemical classes, but are most often organic molecules, e.g., small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

In some embodiments, the candidate agents are proteins. In some aspects, the candidate agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening using the methods herein. The libraries can be bacterial, fungal, viral, and vertebrate proteins, and human proteins.

### B. EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### 1. Example 1

### i. Results

a. Oct1 levels correlate with a stem cell phenotype in primary human tumor cells.

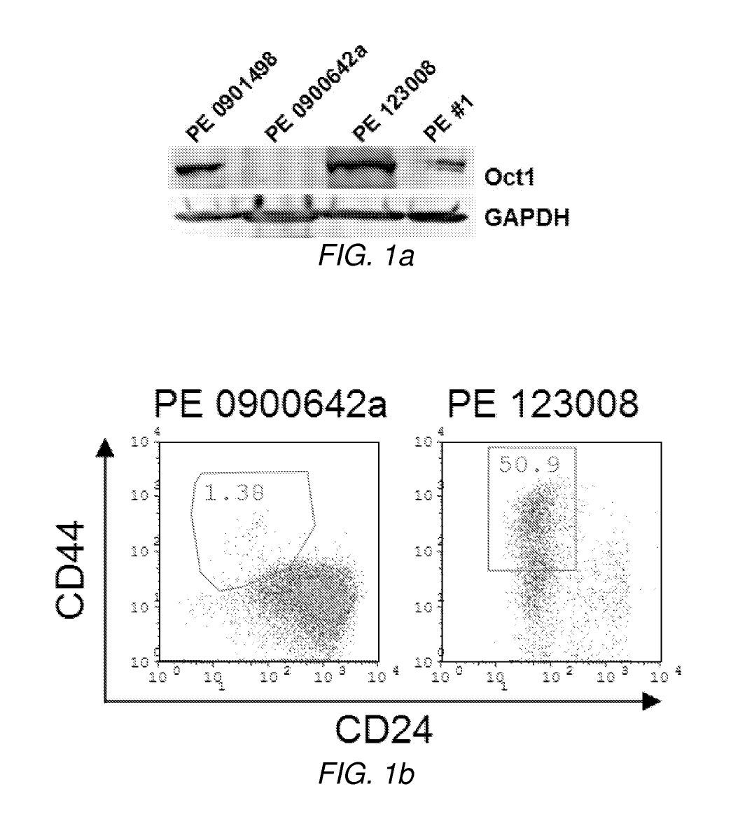

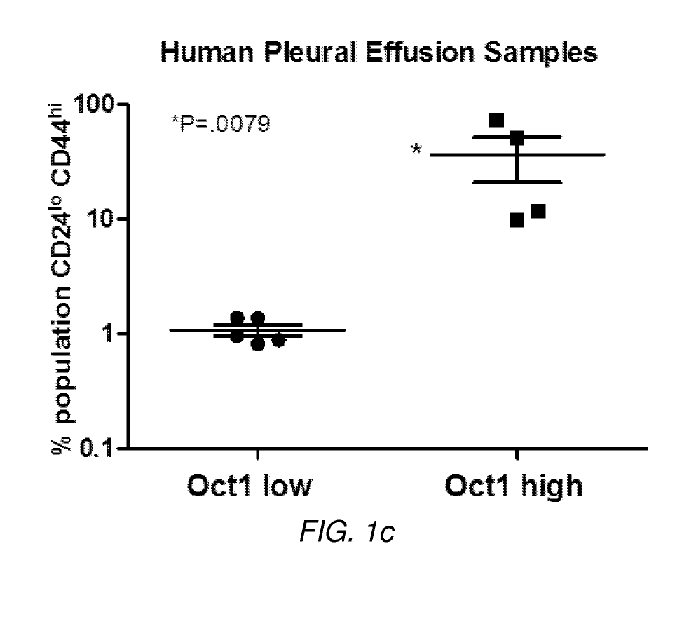

Western blots assaying for Oct1 were performed using primary human metastatic breast carcinoma cells (pleural effusions). Unexpectedly, relative to a GAPDH loading control, Oct1 protein expression was variable (FIG. 1A). It was then determined whether Oct1 levels predicted stem cell content using CD24/44 to detect stem cell populations. Flow cytometry assays were blinded. Pleural effusions with relatively low Oct1 protein levels had low stem cell (CD44^{HI}) contributions (1-2%), whereas those with relatively high Oct1 had higher contributions (10-50%). Examples from each category are shown in (FIG. 1B). Quantification from multiple samples is shown in FIG. 1C. The observed differences were highly significant (p=0.0079).

b. Oct1 RNAi Specifically Decreases ALDH^{Hi} Sub-Populations in Human Tumor Cell Lines.

The data suggested that Oct1 promotes aspects of the stem cell phenotype in somatic cells. To determine whether there was a causal relationship, RNAi was used to reduce Oct1 levels in MB-MDA 231, MCF-7 and A549 breast and lung tumor cell lines, together with the Aldefluor reagent. High ALDH1 activity, as measured by Aldefluor, is a known stem cell marker. It is also a robust marker of tumor stem cells and tumor cell line populations with stem-like properties. Transiently transfected siRNAs and Amaxa nucleofection were used in the cases of MDA 231 and MCF-7. A549 cells were infected with lentiviral particles containing scrambled or Oct1-specific shRNAs (Santa Cruz), and selected using puromycin. Oct1-specific RNAi reduced activity in the main population by approximately two-fold in all cell lines. Oct1-specific RNAi also significantly reduced the number of Aldefluor^{Hi} events relative to a scrambled control (FIG. 2A, C, E). This effect was most easily noted in the A549 cells, in which the Aldefluor^{Hi} “tail” collapses into a more symmetric distribution after Oct1-specific RNAi. Effective RNAi was confirmed using by Western blot (FIG. 2B, D, F).

ChIP assays were conducted using MB-MDA-231 cells and PCR primers spanning the promoter-proximal region of Aldh1a1 (FIG. 2G) to confirm a conserved Oct1 binding site existed in the immediate promoter region of the Aldh1a1 promoter. A signal was observed using Oct1-specific antibodies (FIG. 2H), indicating that Oct1 binds to this promoter in vivo. These data are consistent with a model in which differential Oct1 activity augments Aldh1a1 transcription levels in a sub-group of MB-MDA 231 cells as part of a stem cell program.

c. Oct1 RNAi Specifically Decreases the Dye Efflux-High Side Population in A549 Cells.

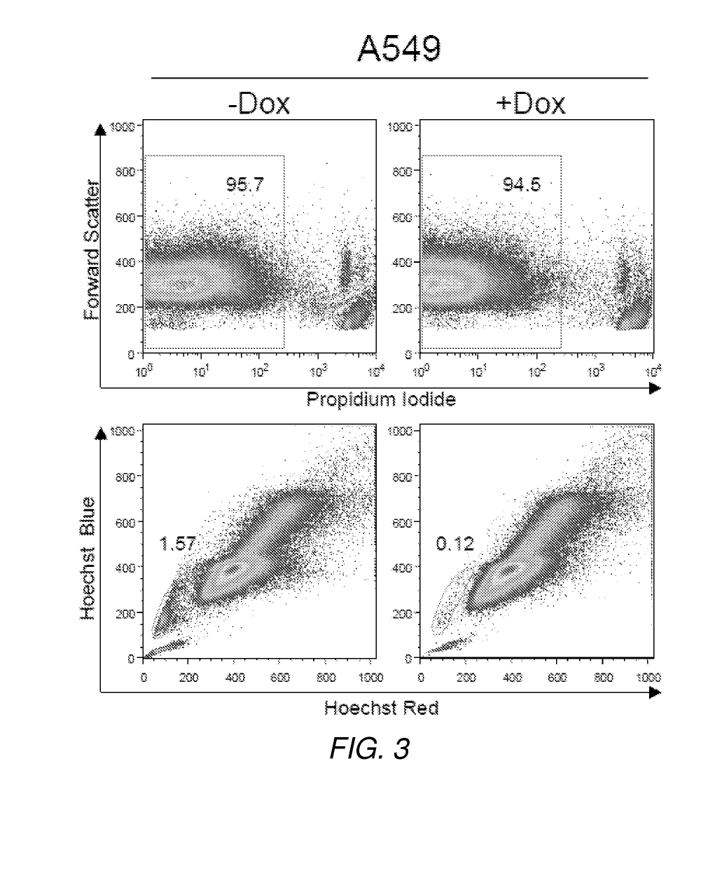

ALDH1 activity is a recently established metric for stem cells. As shown above, Aldh1a1 is also a direct Oct1 target gene. Although Oct1 has specific effects in ALDH1^{Hi} populations, it remained formally possible that Oct1 is important for the expression of the ALDH1 marker only. Stem cells are frequently dye efflux-positive such that Hoechst treatment results in a fraction of cells (the “side population”, SP) with lower steady-state dye levels. A549 cells contain a robust side population. A previously established A549 inducible Oct1 shRNA system was used to determine whether stable knockdown altered the fraction of SP cells. Application of Oct1 RNAi by the addition of doxycycline for four days significantly reduced the SP, while having minimal effects the main population (FIG. 3). The SP was also reduced using the inhibitor verapamil, and no effect was observed using A549 cells engineered with scrambled shRNAs.

d. Oct1 Levels are Elevated in Presumptive Normal and Malignant Colon Epithelial Stem Cells.

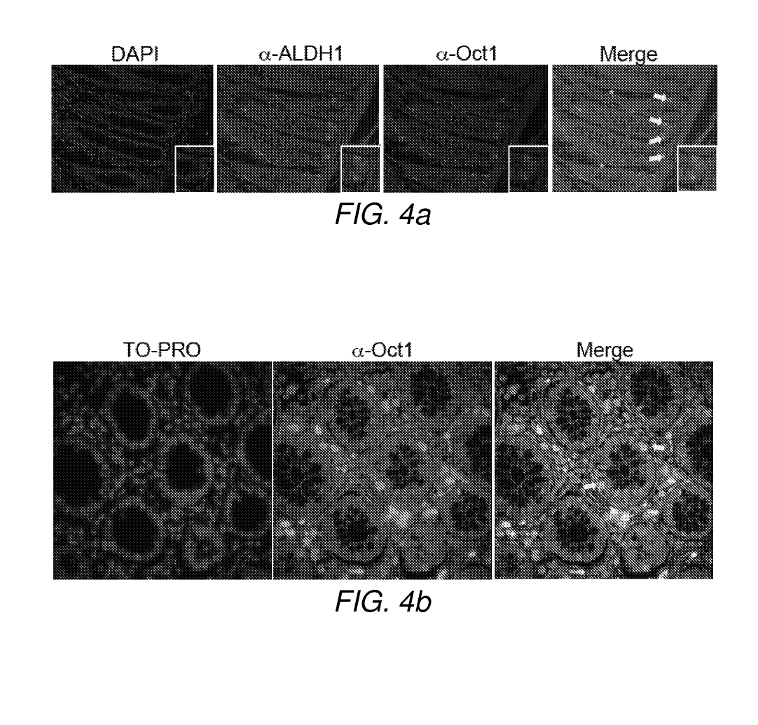

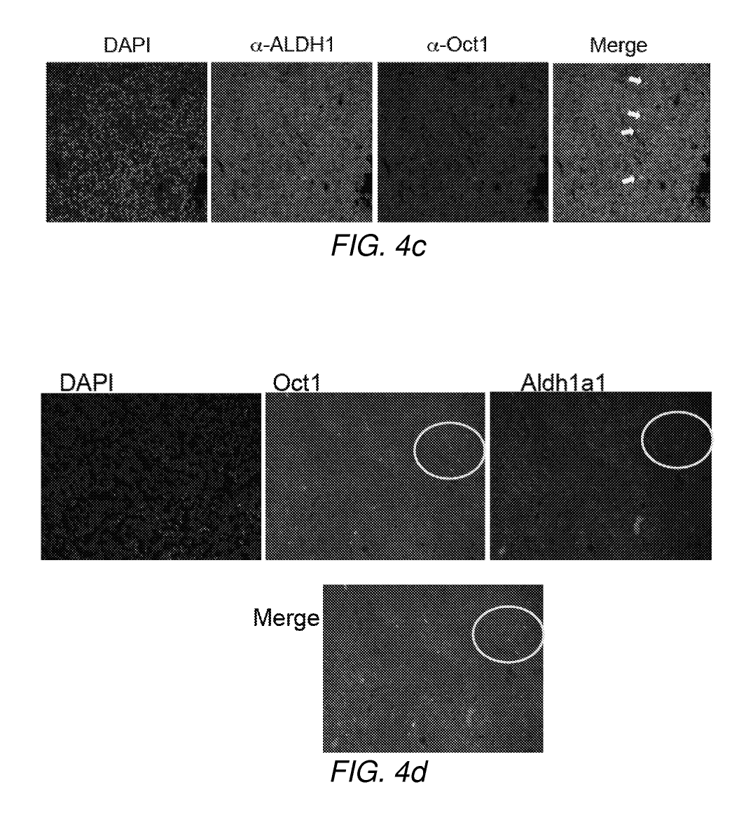

Frozen human colon sections were used together with Oct1 and ALDH1 antibodies in immunofluorescence assays. ALDH1 has been shown to track the stem cell phenotype and also robustly marks tumor stem cells and tumor cell line populations with stem-like properties. Relative to controls lacking the primary antibodies, Oct1 and ALDH1 fluorescence was widely detectable, suggesting that they were both widely expressed at a relatively low level. However, in a subset of cells, some of which were located within gut crypts and others in surrounding stromal cells, more intense staining was evident (FIG. 4A). Merging the fields confirmed that a significant subset of cells at the base of the gut crypts stained strongly for both proteins (arrows). Recent work shows a similar ALDH1 colon staining pattern, and provides evidence that the strongly staining cells are stem cells. A few cells displayed strong expression of only ALDH1a1 (asterisks). Further investigation revealed that intense Oct1 staining could be found in two locations: at the base and also midway up the crypt. An example of the latter is shown in FIG. 4B in which two strongly staining cells were visible (arrows). These cells were clearly within the crypt and not below the lamina propria. Both were one cell removed from the lumen. Multiple such staining patterns were observed but were not always symmetric. Sometimes there were more or fewer cells per crypt, but always one cell removed. These cells may represent a second niche in addition to those at the crypt base. Thus Oct1 was variably expressed in colon epithelium, with stronger staining associated with presumptive somatic stem cells. Similar co-staining was observed in sections of breast epithelium.

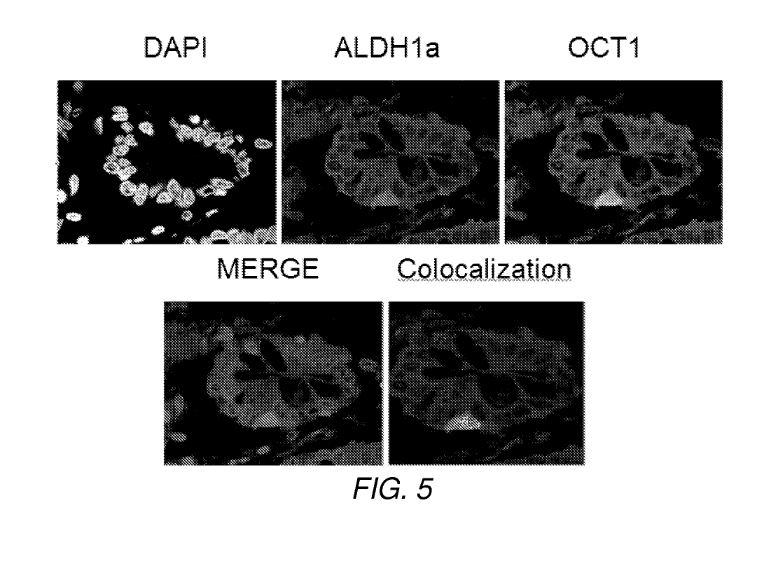

Similar assays were performed on malignant human colon sections. Oct1 and ALDH1a1 staining was also evident in colon (FIG. 4C) as well as breast carcinoma. ALDH1 can also be used to tack malignant colon tumor stem cells. These results suggest that Oct1 levels are elevated in a subset of epithelial tumor stem cells that also stain strongly with ALDH1. Similar results were observed with malignant breast tissue. FIG. 5 shows cells in the colon crypt expressing high Oct1 protein levels also express high levels of the stem cell marker ALDH1a.

e. Oct1 Deficiency is Associated with a Coordinate Decrease in a “Sternness” Gene Expression Signature

Gene expression microarray profiling of Oct1 deficient and littermate control WT primary early-passage MEFs were conducted. Re-analysis using the gene set enrichment algorithm identified a significant alteration in the MELTON_STEMNESS gene expression signature. This gene signature was identified using ES cells, neural stem cells, and hematopoietic stem cells. No fewer than 20 of the 322 genes in this set were significantly altered by Oct1 deficiency, and interestingly all of these were coordinately down-regulated. The most strongly down-regulated genes noted were Diap2, Stam, Gast, Mertk, Laptm4b, Itga6, Zcchc10, Kif2a, Ndufab1, Tgs1 and Chd1. These genes were down-regulated approximately two-fold in Oct1 deficient MEFs, and encode signaling and adhesion molecules, transcriptional regulators, and mitochondrial components, among others. Cross-referencing this group of genes to a Oct1 target gene dataset generated by ChIPseq did not identify any direct targets, suggesting that the altered gene expression was more likely due to an Oct1-controlled stem cell program than a direct consequence of Oct1 transcriptional activation. Several of these genes were tested in the A549 inducible Oct1 shRNA system using qRT-PCR. For most of these genes no change or only small decreases in expression were noted upon Oct1 ablation. However, several showed more dramatic differences.

f. Oct1^{-/-} Cells Are Metabolically Distinct

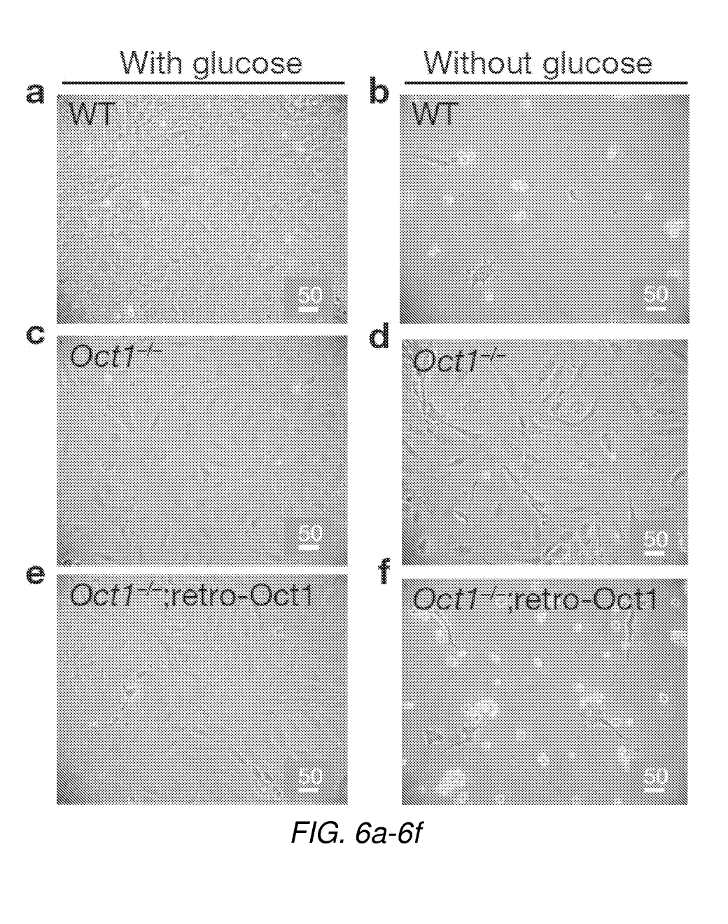

Oct1^{-/-} cells were hypersensitive to stress agents including IR, doxorubicin and hydrogen peroxide. To determine whether Oct1^{-/-} cells were also hypersensitive to metabolic stress, glucose was removed from the medium. Oct1^{-/-} MEFs survived better in the absence of glucose than their wild type (WT) counterparts. WT cells proliferated in high glucose media, but died after 4 days in glucose-free media (FIG. 6a, b). In contrast, Oct1^{-/-} MEFs survived in glucose-free media (FIG. 6d). Oct1 was reintroduced into Oct1^{-/-} cells using retroviral transduction and cultured the cells in media lacking glucose. Oct1^{-/-} cells with ectopic Oct1 expression survived in high glucose media, but were sensitive to glucose withdrawal (FIG. 6e, f). These results indicated that Oct1-deficiency caused cellular changes that confered resistance to glucose withdrawal.

Next, ATP levels in Oct1^{-/-} and WT MEFs were measured using a luciferase-based assay. Oct1^{-/-} cells had significantly higher ATP pools compared to WT (FIG. 6g). The NAD⁺/NADH ratio was another important indicator of cellular energetic status. NAD⁺/NADH was significantly reduced in Oct1^{-/-} MEFs (FIG. 6h). A Clark-type oxygen electrode was used to measure O₂ consumption in primary MEFs and embryos in the presence and absence of the mitochondrial proton decoupler 2,4-dinitrophenol (2,4-DNP). This reagent eliminated the proton gradient across the mitochondrial inner membrane, and in an attempt to re-establish the gradient, maximum aerobic capacity was displayed. Although little change was identified in untreated MEFs, a significant increase was present using 2,4-DNP (FIG. 6i). In the embryos, O₂ consumption was higher in the Oct1^{-/-} condition (FIG. 6j; gray bars). 2,4-DNP accentuated this effect (black bars). The mitochondrial membrane potential (ΔΨₘ) was also examined using the JC-1 dye and flow cytometry. JC-1 accumulates in mitochondria in an ΔΨₘ-dependent manner, where the J-aggregates (dimers of JC-1 dye) fluoresce red. ΔΨₘ was increased approximately 1.5-fold in Oct1^{-/-} MEFs (FIG. 6k).

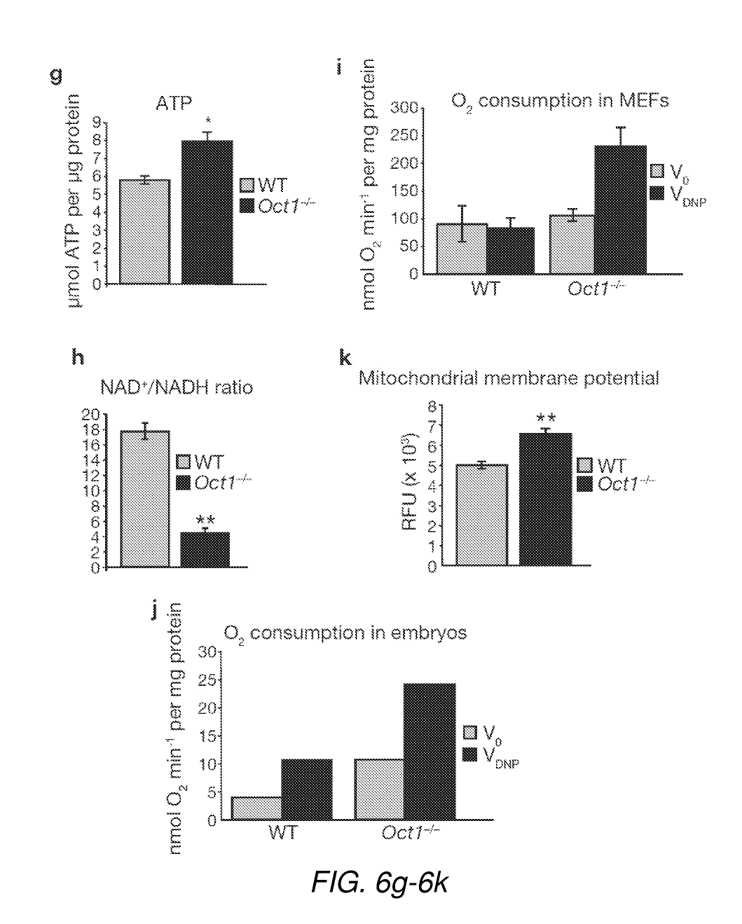

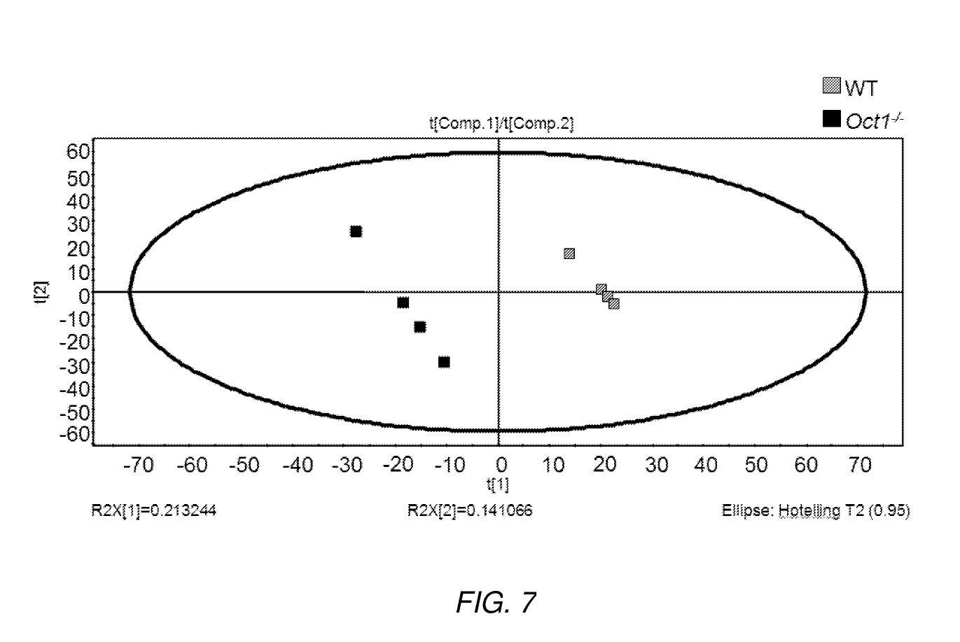

To objectively determine the metabolic changes induced by loss of Oct1, a metabolomic analysis of WT and Oct1^{-/-} MEFs was performed. These experiments provided a snapshot of steady-state metabolite levels, which may be a product of altered synthesis, degradation, or both. Cells were collected in cold wash buffer and snap frozen in liquid nitrogen to preserve the metabolic state. Metabolites were analyzed by gas chromatography/mass spectroscopy (GC/MS) for accurate identification and quantification of small metabolites (see Methods). Sample processing for GC/MS analysis often results in loss of phosphate groups such that ATP/ADP/AMP or glucose 6-phosphate were identified as adenylate or glucose, respectively. Four experimental replicates of WT and Oct1^{-/-} MEFs were used. A total of 1296 peaks were identified, representing both metabolites and their degradation products. Principal component analysis (PCA) and partial least squares projections to latent structures (PLS) were applied to the datasets to simplify the multivariate data in an unbiased manner. PCA and PLS (FIG. 7) clustered the samples into distinct groups representing WT and Oct1^{-/-} MEFs, indicating that their metabolic profiles were significantly different.

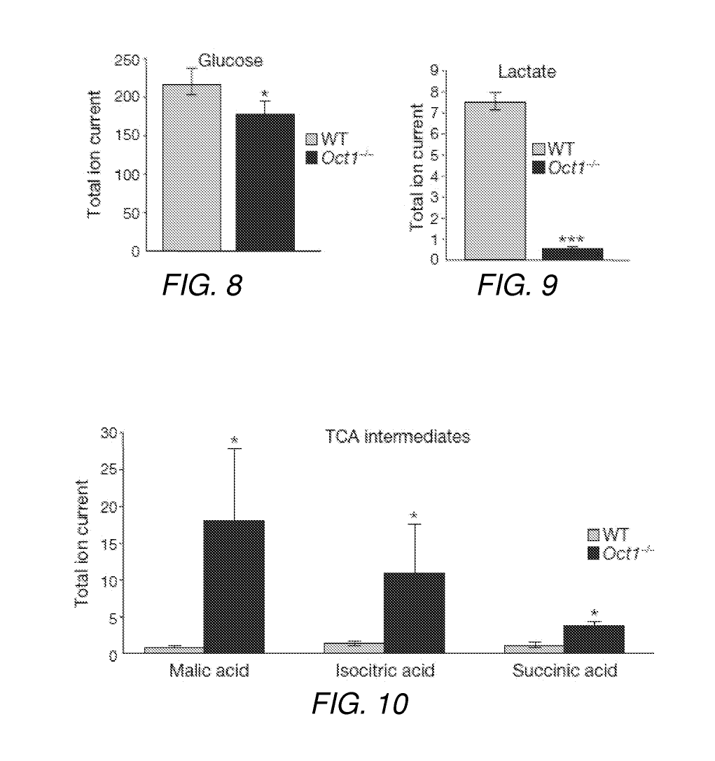

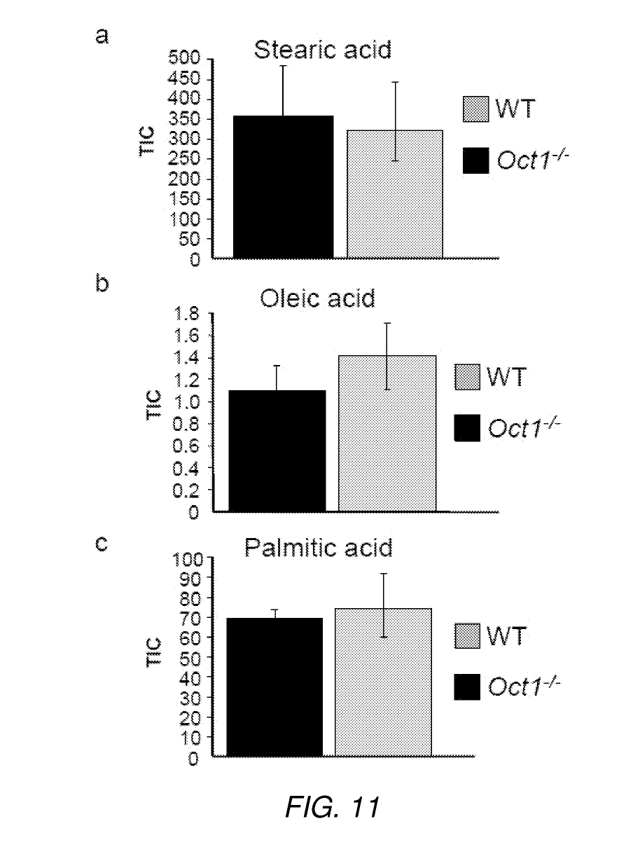

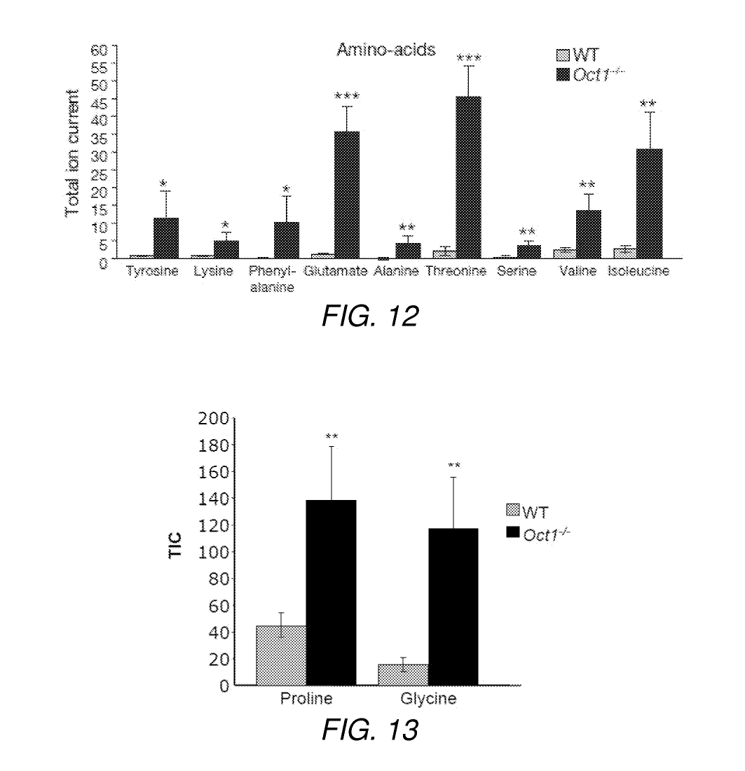

Sixty specific metabolites were identified, most of which were not significantly altered. Glucose and lactate were significantly lowered in Oct1^{-/-} cells (FIGS. 8 and 9). The change in lactate levels (>seven-fold, P<0.001) was particularly prominent. TCA cycle intermediates including malate, isocitrate and succinate were elevated in Oct1^{-/-} MEFs (FIG. 10). These data were consistent with augmented mitochondrial function. Although there was no change in the cellular levels of free fatty acids (FIG. 11), levels of many amino acids including glutamate, threonine and isoleucine were elevated in the Oct1^{-/-} cells (FIG. 12). Increased levels of steady-state proline and glycine were also observed (FIG. 13). In contrast, arginine and cysteine levels were unaltered. Oct1^{-/-} MEFs also had higher creatinine and urea levels (FIG. 14), suggesting increased amino acid catabolism.

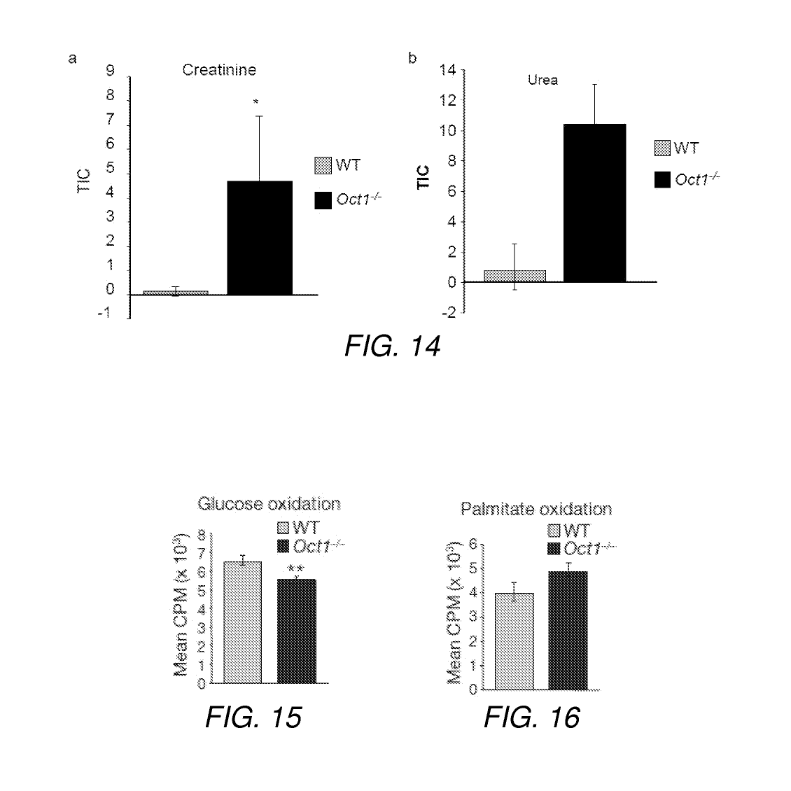

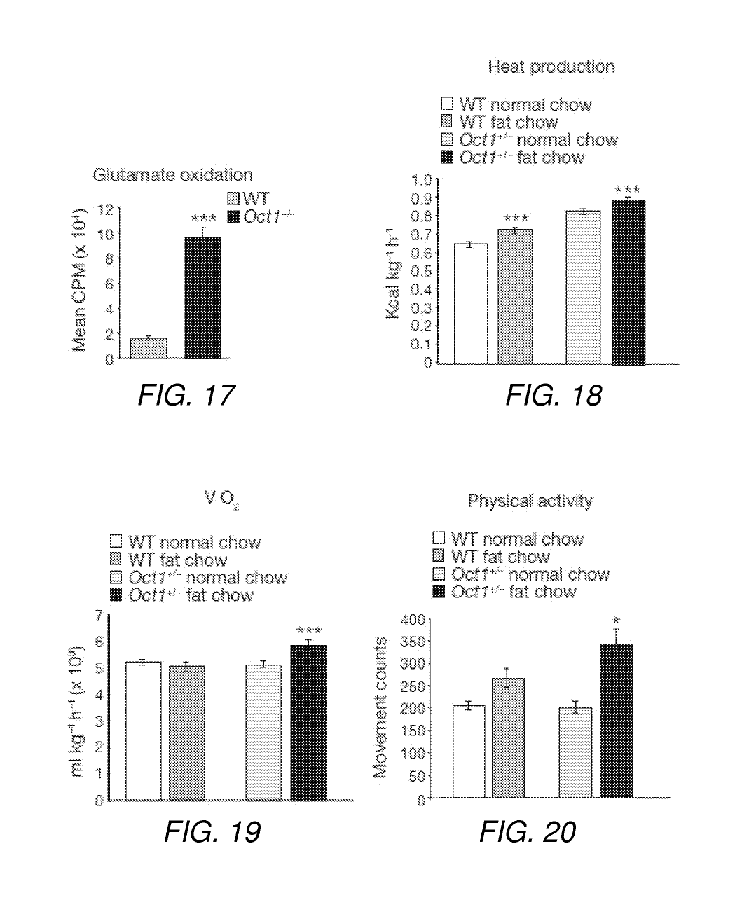

To assess glucose, fatty acid and amino acid oxidation, MEFs were cultured in media containing ¹⁴C-labeled glucose, ³H-labeled palmitic acid or ¹⁴C-labeled glutamate. ¹⁴CO₂ and ³H₂O were trapped and radioactivity counted by scintillation. Consistent with other findings herein that Oct1^{-/-} MEFs are resistant to glucose withdrawal, decreased glucose oxidation was observed in Oct1^{-/-} cells relative to WT (FIG. 15). Palmitate oxidation showed little change (FIG. 16). Remarkably, there was a robust increase in glutamate oxidation (FIG. 17). These results suggested that Oct1^{-/-} MEFs preferentially oxidize amino acids for energy production.

g. Oct1^{+/-} Mice are More Metabolically Active

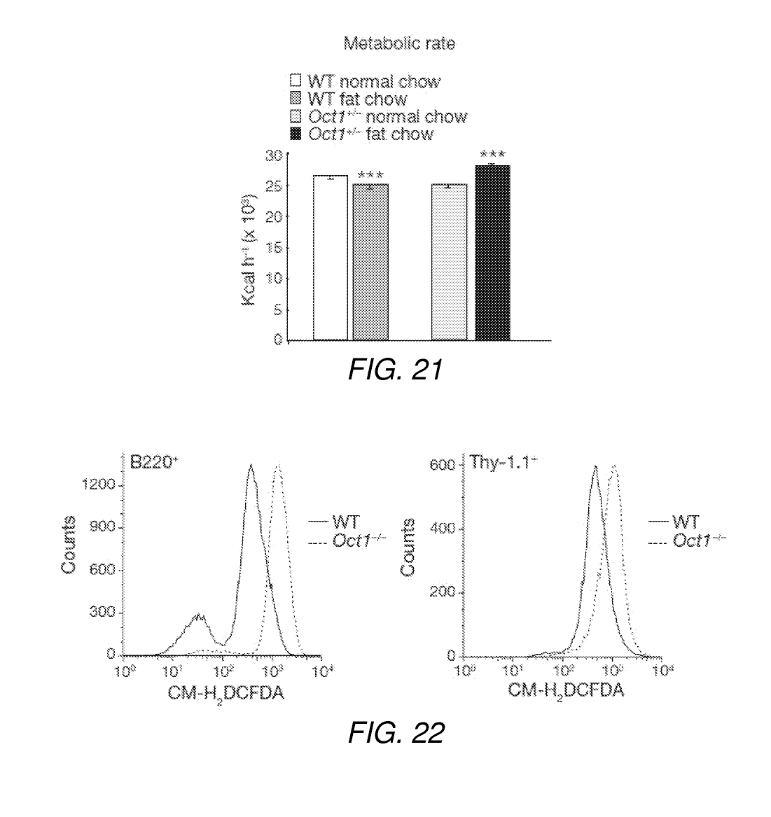

Although Oct1^{-/-} mice died in utero, Oct1^{+/-} mice survived and did not show any obvious abnormalities. Because Oct1 levels and activity in the heterozygous state are half of that in wild type, metabolism was investigated in Oct1^{+/-} mice. Oct1^{+/-} mice fed high fat diets were more metabolically active, having higher heat production, oxygen usage, physical activity and metabolic rates than their wild-type littermates (FIGS. 18-21)

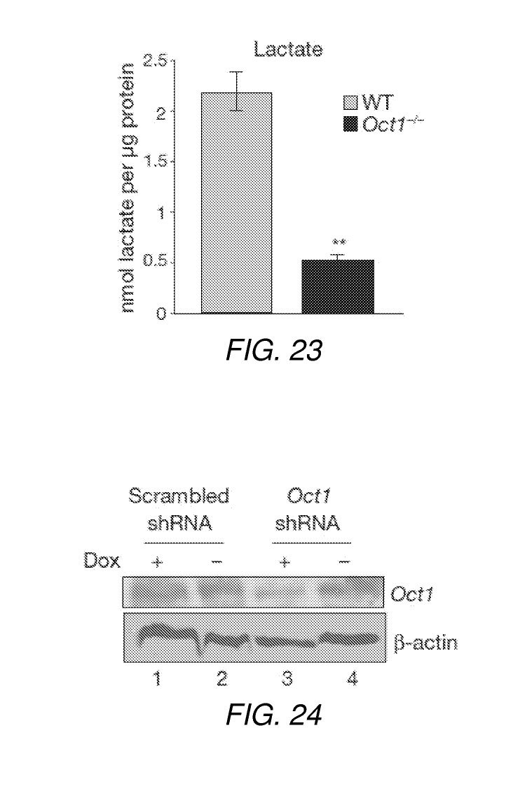

To assess the behavior of adult Oct1^{-/-} B and T lymphocytes, hematopoietic progenitors from Oct1^{-/-} embryos were transplanted into irradiated rag1^{-/-} mice. Mice were sacrificed after 10 weeks and splenic WBCs were harvested and stained with CM-H₂DCFDA and antibodies to B220 and thy-1 to determine ROS levels in Oct1^{-/-} B and T lymphocytes. Oct1^{-/-} lymphocytes showed increased ROS production compared with WT (FIG. 22). Oct1^{-/-} WBCs also displayed significantly decreased lactate (FIG. 23).

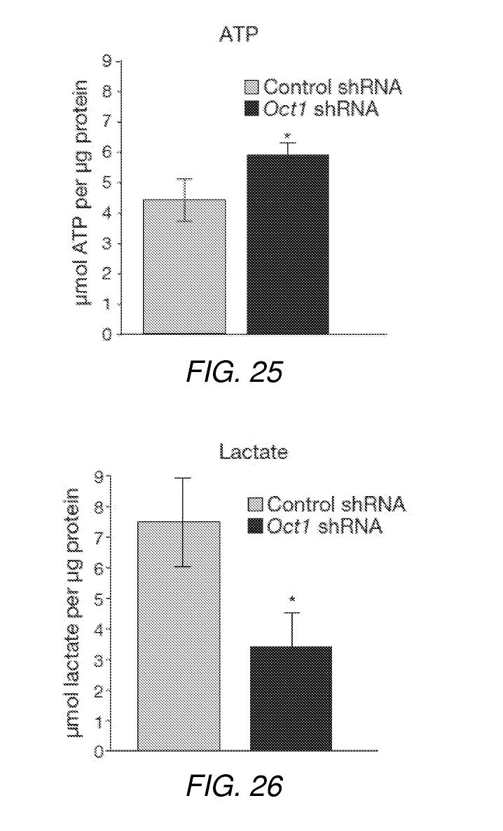

To assess acute as well as germline Oct1 ablation, and to extend these results to human cells, tetracycline-inducible shRNAs were used together with A549 lung adenocarcinoma cells. The siRNA produced from the shRNA is as follows: 5′GCCTTGAACCTCAGCTTTAAG3′ (SEQ ID NO:2). Using this tet-ON system, there was a significant knock-down of Oct1 using specific but not scrambled shRNAs (FIG. 24). Oct1 shRNA expression decreased lactate and increased ATP levels compared to scrambled shRNA (FIGS. 25 and 26). The effect of Oct1 overexpression on cellular metabolism was also examined. A549 cells were transiently transfected with control and Oct1 expression plasmids. ATP levels decreased, whereas lactate level increased in the cells transfected with Oct1 plasmid (FIG. 27).

h. Oct1 Regulates Targets Upstream of the Metabolic Changes

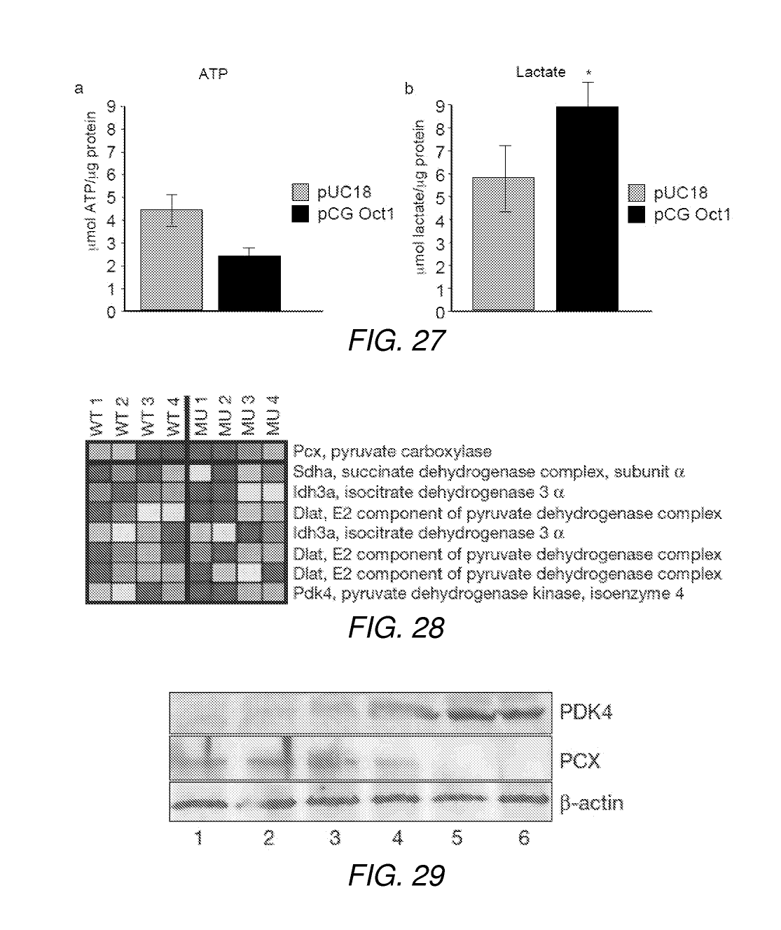

Expression microarray profiling of WT and Oct1^{-/-} early-passage MEFs were previously conducted. Re-analysis of the gene expression data using GSEA (Gene Set Enrichment Analysis, http://www.broad.mit.edu/GSEA) revealed additional altered gene expression signatures. Three of these were “Krebs-TCA Cycle”, “Glutamate Metabolism” and “Alanine and Aspartate Metabolism”. Within the set there were both up- and down-regulated genes (FIG. 28), encoding positive and negative TCA cycle regulators.

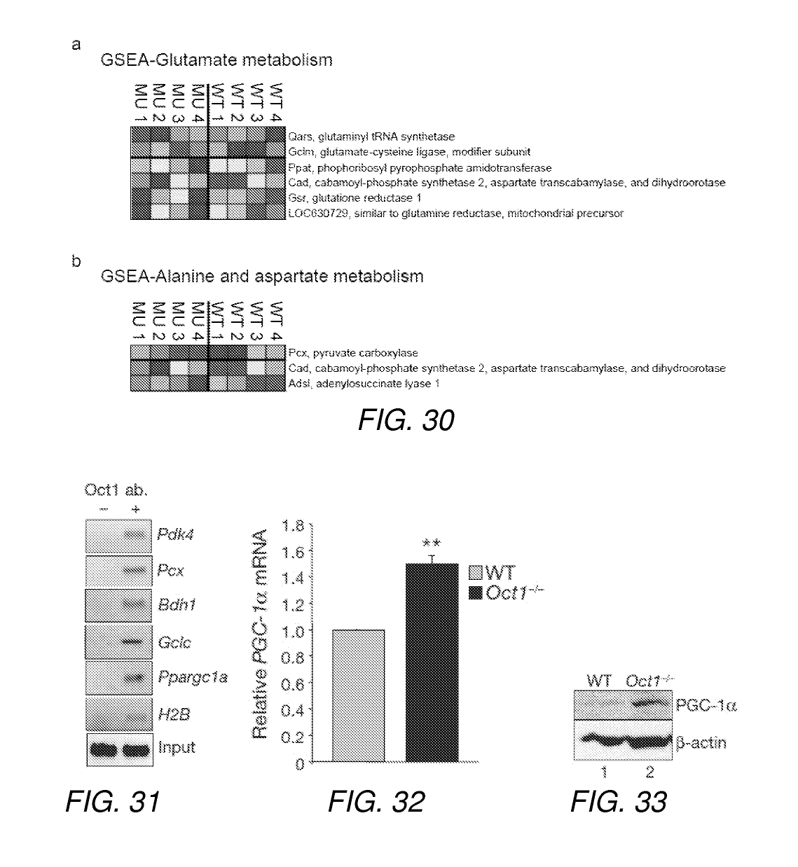

The most significantly down-regulated gene in the Krebs-TCA cycle set encoded pyruvate carboxylase (PCX), which catalyzes the conversion of pyruvate to the TCA intermediate oxaloacetate. The most significantly up-regulated gene was pyruvate dehydrogenase kinase 4 (Pdk4), an inhibitor of pyruvate dehydrogenase (PDH), which is responsible for the first step in converting pyruvate to acetyl-CoA. For pyruvate to enter the TCA cycle, either the PCX or PDH routes must be used. The data were consistent with the finding that glucose oxidation is decreased in the Oct1 knockout. Surprisingly, Dlat, which encodes part of the E2 component of the pyruvate dehydrogenase complex, was increased in Oct1^{-/-} MEFs. This increase may represent an attempt at compensation and/or feedback control. Western blotting confirmed the altered PCX and PDK4 levels in Oct1^{-/-} cells (FIG. 29). Isocitrate and succinate dehydrogenase levels were also elevated. It was postulated that in Oct1^{-/-} cells, amino acids supply both the intermediates and carbon to an augmented TCA cycle. This model was consistent with the increased amino acid steady-state levels and oxidation, increased steady-state levels of TCA intermediates, and increased mitochondrial function in Oct1^{-/-} cells. Gene expression profiling showed alterations in genes involved in amino acid metabolism, supporting this model (FIG. 30).

Regions of genes encoding regulators of metabolism from known Oct4 targets in ES cells were identified, hypothesizing that they may be Oct1 targets in somatic cells. Oct4 is homologous to Oct1 through much of its length and the two proteins share nearly identical DNA binding specificity. Furthermore, ES cells are highly glycolytic and have low levels of mitochondrial oxygen consumption. Notably, both Pcx and Pdk4 were identified as transcriptional targets in ES cells, though only Pcx as a direct Oct4 target. Primers spanning these bound regions were used in chromatin immunoprecipitation (ChIP) assays, identifying bound Oct1 in both cases (FIG. 31). Two other genes, involved in glutathione biosynthesis (Gclc) and fatty-acid metabolism (Bdh1), were identified as likely Oct1 targets in a previous study. ChIP assays indicated that these were Oct1 targets. In addition, sequence inspection of Ppargc1a gene which encodes peroxisome proliferator-activated receptor-γ coactivator-1 alpha (PGC-1α) identified a conserved close match to an octamer sequence 366 by upstream of TSS. Ppargc1a is also a direct Oct1 target (FIG. 31). PGC-1α protein levels were increased in Oct1^{-/-} MEFs (FIGS. 32 and 33).

i. Increased Mitochondrial Density in Oct1^{-/-} MEFs

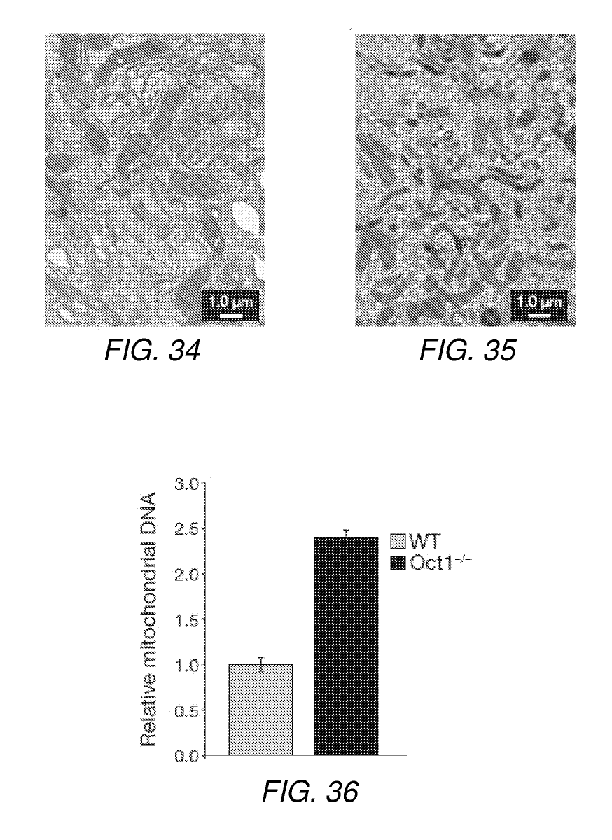

PGC-1α is a key regulator of mitochondrial biogenesis. Because PGC-1α levels were increased in Oct1^{-/-} MEFs, transmission electron microscopy (TEM) was employed to visualize mitochondria in these cells. Increased mitochondrial density was observed at 3,900× (FIGS. 34 and 35). At higher magnification, ultrastructural differences were also apparent, especially in the structure and organization of mitochondrial cristae. More electron transport chain (ETC) complexes were also apparent, as measured using Western blotting. The relative abundance of mitochondrial DNA with respect to nuclear DNA in WT and Oct1^{-/-} MEFs was determined by quantitative PCR. Oct1^{-/-} MEFs showed >two-fold higher mitochondrial DNA content compared to WT (FIG. 36). These results suggested that the increased ATP levels and O₂ consumption in Oct1^{-/-} MEFs were a result of increased mitochondrial density and activity.

j. Loss of Oct1 Decreases Transformation and Tumor Potential

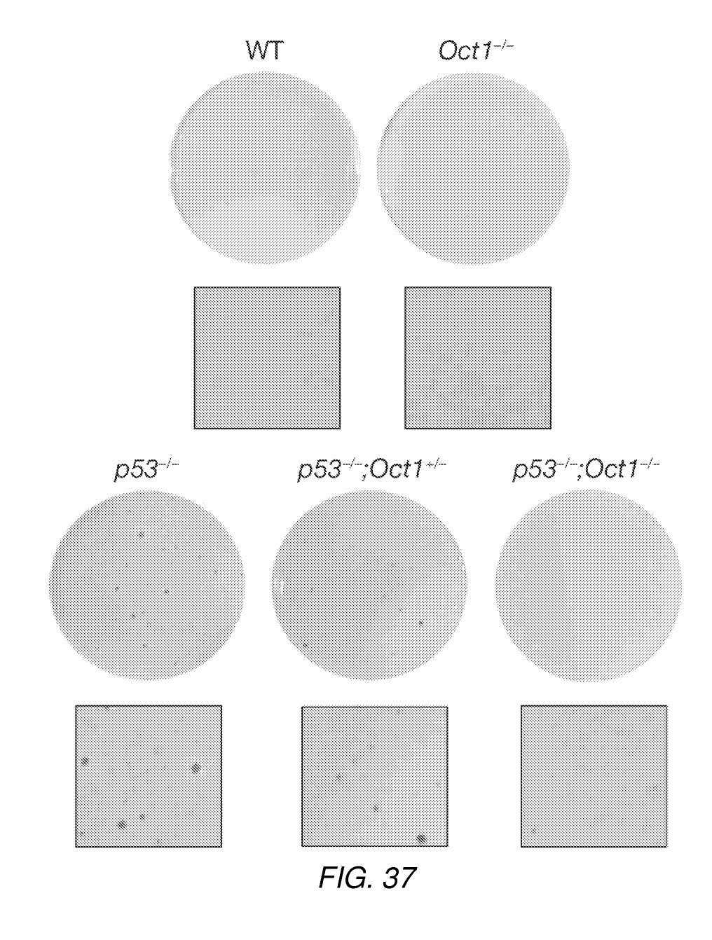

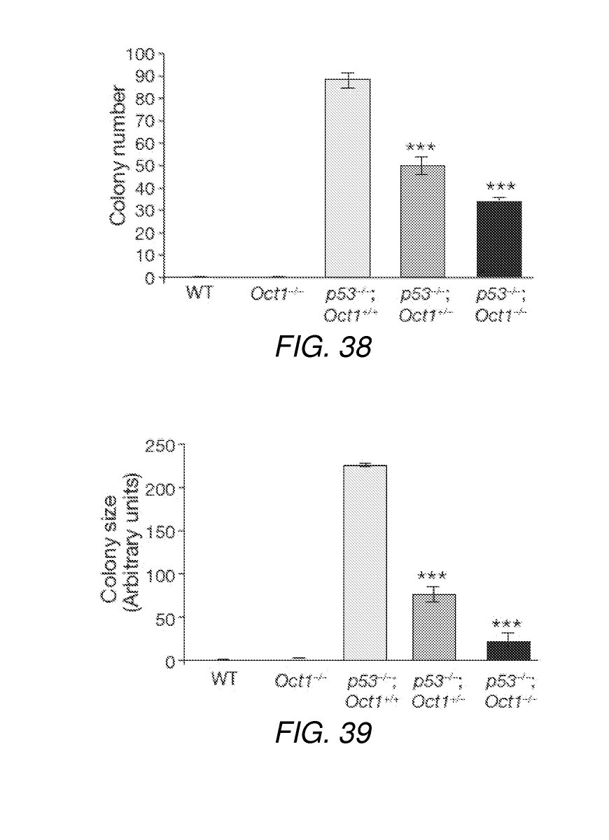

Oct1^{-/-} cells displayed increased oxidative metabolism and produced less lactate, properties associated with anti-cancer effects. Soft agar colony assays were conducted to test the effect of loss of Oct1 on transformation. Transduction of MEFs with H-Ras containing an activating Val12 mutation resulted in senescence and the absence of visible soft agar colonies. In contrast, fibroblasts lacking p53 did not undergo senescence and infection with H-RasV12 results in soft agar colonies. MEFs harboring varying Oct1 levels were infected with retroviruses expressing H-RasV12. WT and Oct1^{-/-} cells transduced with H-RasV12 did not form colonies while p53^{-/-} MEFs form numerous large colonies (FIGS. 37 and 38). p53^{-/-}; Oct1^{+/-} MEFs formed smaller and fewer colonies. Most strikingly, p53^{-/-}; Oct1^{-/-} MEFs formed soft agar colonies poorly (FIGS. 37-39). Microscopic inspection of the agars showed only a few rounds of division in the microcolonies that were formed.

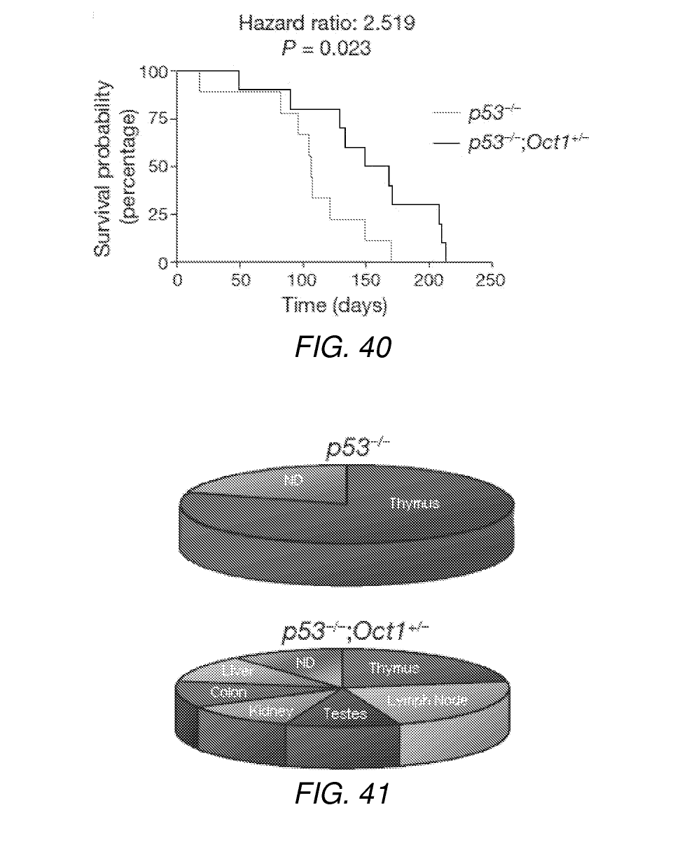

The issue of whether Oct1 can control tumorigenicity in vivo was addressed by crossing Oct1^{+/-} mice to the well-characterized p53 mouse model. p53 regulates mitochondrial function, suggesting a potential interaction between these two transcription factors (i.e., Oct1 and p53). Because loss of a single Oct1 allele can significantly reduce the transformation potential of p53^{-/-} MEFs and induce metabolic changes in adult mice, Oct1 heterozygosity was predicted to produce anti-cancer effects. Oct1^{+/-} and p53^{-/-} mice were intercrossed and tumor incidence and spectrum of p53^{-/-}; Oct1^{+/-} mice were compared to p53^{-/-}. Oct1^{+/-}; p53^{-/-} C57BL/6 mice survived significantly longer than p53^{-/-} (hazard ratio=2.5, FIG. 40). C57BL/6 p53^{-/-} mice primarily developed thymic lymphoma (FIG. 41). Interestingly, the tumor spectrum of Oct1^{+/-}; p53^{-/-} mice included several other organs including a large lymph node contribution (FIG. 41). Recipient mice repopulated with Oct1-deficient lymphoid systems had normal T-cell development and thymic cell counts, decreasing the likelihood that thymic lymphoma would be suppressed in C57BL/5 p53^{-/-}; Oct1^{+/-} animals was due to reduced or abnormal thymi.

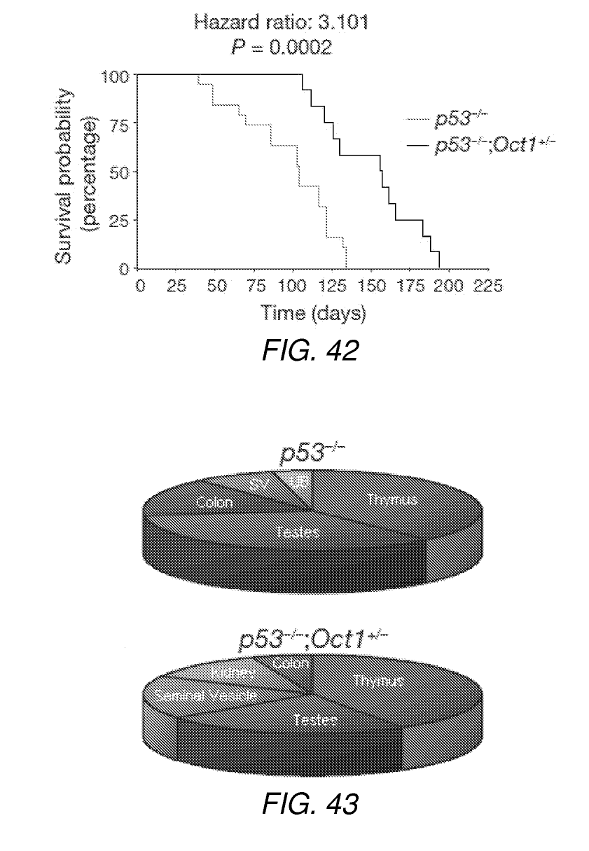

Identical studies were performed on a 129 background. The results in this case were more robust (hazard ratio=3.1, FIG. 42). 129 p53^{-/-} mice primarily developed thymic lymphoma and testicular tumors (FIG. 43). In the p53^{-/-}; Oct1^{+/-} condition the tumor spectrum was more mildly altered, most notably by the emergence of tumors in the kidneys (FIG. 43).

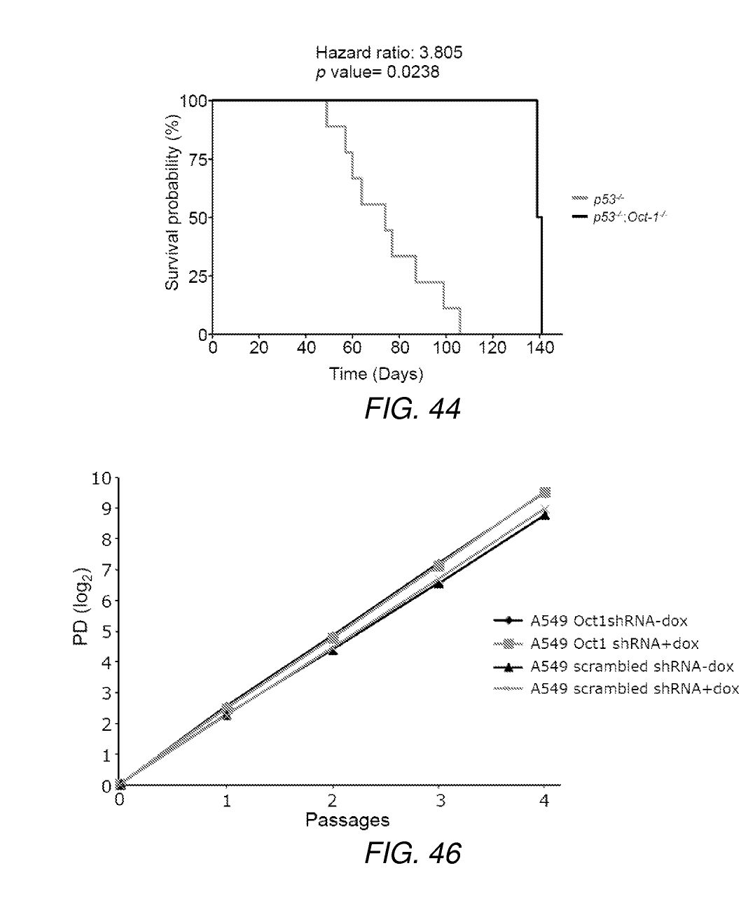

To study whether the anti-tumor properties of Oct1 deficiency were cell autonomous, liver cells were harvested from p53^{-/-} and p53^{-/-}; Oct1^{-/-} embryos and transplanted them into irradiated rag1^{-/-} mice. The tumor-free life-span of recipient mice transplanted with p53^{-/-}; Oct1^{-/-} cells was significantly longer than that of p53−/− (FIG. 44).

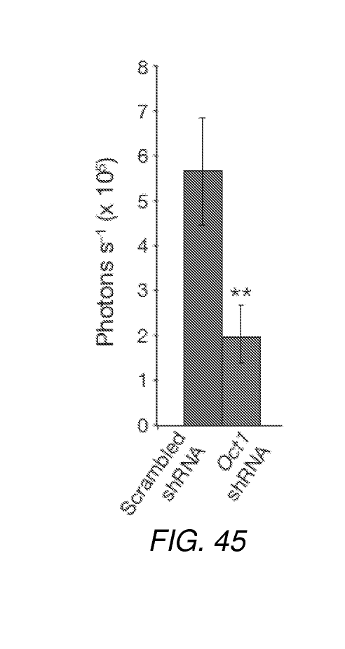

The tumor-forming potential of A549 human lung carcinoma cells was also tested by expressing tetracycline-inducible scrambled and Oct1 shRNAs. These cells constitutively expressed luciferase, allowing the tumor to be visualized by bioluminescence. 2×10⁶ cells were injected in the flanks of nude mice. Doxycycline was provided in the drinking water. At three weeks, luminescence in Oct1 shRNA expressing cells was significantly lower than that observed using the scrambled shRNA (FIG. 45). The growth rates of these cells in culture were comparable (FIG. 46).

k. Metabolic-Alterations Underlie Oct1's Effect on Transformation Potential

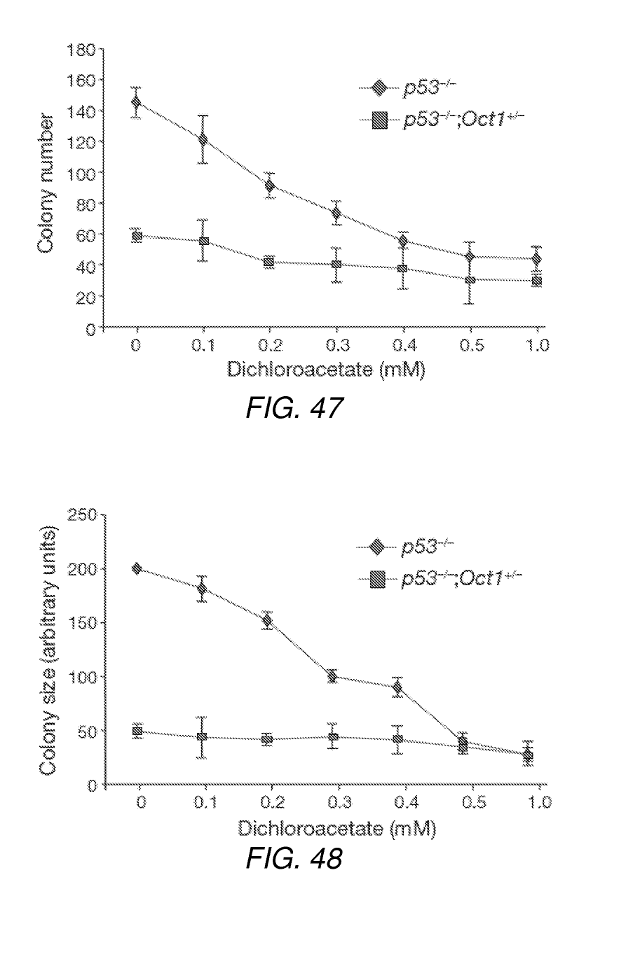

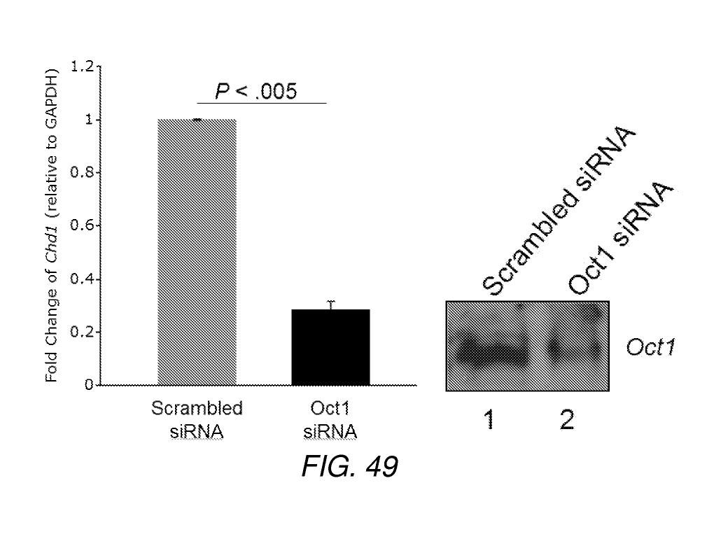

Dichloroacetate (DCA) is an inhibitor of pyruvate dehydrogenase kinase (PDK), which inhibits conversion of pyruvate to acetyl CoA. The effect of DCA on the transformation potential of p53^{-/-} and Oct1^{+/-}; p53^{-/-} MEFs infected with retroviruses expressing H-RasV12 was determined. Oct1 heterozygous MEFs were used, as colony number and size are already strongly reduced in the Oct1-deficient condition (FIGS. 47 and 48). DCA decreased average colony number and size in p53^{-/-} MEFs in a dose-dependent manner. In Oct1^{+/-}; p53^{-/-} MEFs, DCA had little effect on either colony number or size (FIGS. 47 and 48). These findings were consistent with a model in which Oct1 and DCA operate in the same pathway to control transformation.

### ii. Materials and Methods

a. Cell Culture

E12.5 MEFs were generated. MEFs, A549 human lung carcinoma cells, MCF-7 human breast adenocarcinoma cells, and MB-MDA-231 human breast adenocarcinoma cells were obtained from ATCC and maintained in DMEM supplemented with 10% fetal bovine serum, 6 mM L-glutamine, 50 U/ml penicillin, 50 mg/ml streptomycin and 50 mM b-mercaptoethanol (Sigma) at 37° C. and 5% CO₂ in a humidified atmosphere. For glucose deprivation, serum concentration was reduced to 2.5% and D-glucose, sodium pyruvate and supplemental L-glutamine were omitted.

ER−, PR−, HER2+ human pulmonary effusion cells and ER−, PR−, HER2− xenograft cells were maintained in DMEM/F12 1:1 supplemented with 10 mM Hepes, 5% fetal bovine serum, 1 mg/ml bovine serum albumin, 1 mg/ml insulin with transferring/selenium, 0.5 mg/ml hydrocortisone and 50 mg/ml gentamycin. These cells were maintained in 5% CO₂ and air in a humidified 37° C. incubator.

b. Flow Cytometry

The cells were plated on plastic for 6 hr to allow epithelial cells to adhere and deplete hematopoietic cells. Non-viable and lin-positive (CD2, CD3, CD10, CD16, CD18, CD31, CD64, CD140b) cells were gated out. Cells were also stained with CD24 and CD44 antibodies and processed.

c. ChIP

Chromatin immunoprecipitation was performed as described previously. See Boyd K E and Farnham P J. Coexamination of site-specific transcription factor binding and promoter activity in living cells. Mol. Cell. Biol. 19: 8393-8399 (1999). Primer sequences used for ChIP were as follows: Aldh1a1 for, 5′TGCTCCAGCATCGAATTTGTAC3′ (SEQ ID NO: 23); rev, 5′AGAGCAGCTGCTGCTGCATACACTT (SEQ ID NO: 24).

d. Aldefluor

Aldehyde dehydrogenase activity was measured in cells as described previously using the Aldefluor kit (Stem Cell Technologies) with 125 ng ALDH substrate and 100 mM DEAB (Sigma-Aldrich).

e. Hoechst Side Population Assay

Side population assays were performed as described previously, with the following modifications: the dye incubation was performed in DMEM containing 10% FBS, and the buffer for flow cytometry was PBS with 1 mM EDTA and 0.5 mM EGTA.

f. Oligonucleotides

The sequences of all oligonucleotides are provided in Table 2.

TABLE 2
Sequence of Oligos
Scrambled and Oct1 shRNAs

| | | |
|---|---|---|
| Scrambled shRNA | GGAATTAATTGCATGAATTAG | SEQ ID NO: 1 |
| Oct1 shRNA | GCCTTGAACCTCAGCTTTAAG | SEQ ID NO: 2 |

Sequence of primers used for RT-PCR analysis
of mitochondrial DNA

| | | |
|---|---|---|
| b-actin-F | TGTTACCAACTGGGACGACA | SEQ ID NO: 3 |
| b-actin-R | CTATGGGAGAACGGCAGAAG | SEQ ID NO: 4 |
| mt CytB-F | AACATACGAAAAACACACCCATT | SEQ ID NO: 5 |
| mt CytB-R | AGTGTATGGCTAAGAAAAGACCTG | SEQ ID NO: 6 |

Primers used for Chromatin immunoprecipitation

| | | |
|---|---|---|
| Pcx-F | CAGACCCCAGGTGGTACCGG | SEQ ID NO: 7 |
| Pcx-R | TAACAGATGCACGGGGGTTG | SEQ ID NO: 8 |
| Bdh1-F | CTTCCCTGTTGAGTTGGCCC | SEQ ID NO: 9 |
| Bdh1-R | CAAGCTGGAGCTAAATAAGC | SEQ ID NO: 10 |
| Pdk4-F | ATCCCAGTTCACTTCTCTCCTG | SEQ ID NO: 11 |
| Pdk4-R | GCAAACTAGAAGGCCTTAGAG | SEQ ID NO: 12 |
| Gclc-F | CTAATCTGGTATCCCCCGAGTCAC | SEQ ID NO: 13 |
| Gclc-R | CCGGGACACTTTTACATACATTTG | SEQ ID NO: 14 |
| Ppargc1a-F | CCCTGCTCACATAATAACTCAAATC | SEQ ID NO: 15 |
| Ppargc1a-R | GGGGCTACTTGGAAACCATTTC | SEQ ID NO: 16 |
| H2B-F | CAATGGAAAGCGATTATAGCAACAAG | SEQ ID NO: 17 |
| H2B-R | GGACTTCGCAGGCTCAGGCATAG | SEQ ID NO: 18 |

Quantification of mRNA levels by real-time PCR
for PGC-1a

| | | |
|---|---|---|
| PGC-1α-F | AACCACACCCACAGGATCAGA | SEQ ID NO: 19 |
| PGC 1α-R | TCTTCGCTTTATTGCTCCATGA | SEQ ID NO: 20 |
| β-actin-F | TGCTCCCCGGGCTGTAT | SEQ ID NO: 21 |
| β-actin-R | CATAGGAGTCCTTCTGACCCATTC | SEQ ID NO: 22 |

g. Metabolic Measurements

For intracellular ATP, MEFs were collected and lysed with 1 M perchloric acid. Debris was removed by centrifugation. The supernatant was neutralized using 1 M KOH and the precipitate cleared by centrifugation. An ATP assay kit (Promega) was used. Luminescence was measured using a microplate reader (Perkin Elmer) and normalized to protein concentration. NAD+/NADH ratios were measured using a kit (Biovision).

For O₂ consumption, 1.2×10⁶ fibroblasts or embryos suspended in TD buffer (137 mM NaCl; 5 mM KCl; 0.7 mM Na₂HPO₄; 25 mM Tris-HCl, pH 7.4) were placed in an airtight chamber and stirred continuously at 37° C. A Clark-type (FOXY-R) electrode monitored dissolved O₂ over time. O₂ in the buffer was detected using a USB 4000-FL spectrophotometer and analyzed using the OOI Sensors program (Ocean Optics). 2,4-DNP was added to 83 μM when respiration reached a stable rate to measure maximal respiration.

For ΔΨₘ, WT and Oct1^{-/-} MEFs were stained with JC-1 (Stratagene). Cells were resuspended in assay buffer and analyzed by flow cytometry using a Becton Dickinson FACSCalibur and CellQuest software.

h. GC/MS

Cells were collected in 100 μl of ice-cold buffer (1 mM sodium phosphate, pH 7.4; 150 mM NaCl) and snap frozen in liquid nitrogen. Pellets were extracted with 900 μl methanol. Samples were vortexed for 1 min and sonicated for 5 min at 70° C. The vortex/sonication cycle was repeated. Debris were removed by centrifugation at 5000 g. The supernatant was dried en vacuo and resuspended in 100 μl pyridine containing 20 mg/ml O-methoxyamine hydrochloride and 0.03 mg/ml methyl stearate (Sigma) as internal standards. This solution was vortexed for 1 min and incubated for 1.5 hr at 30° C. 50 μl N-methyl-N-trimethylsilyltrifluoroacetamide containing 1% trimethylchlorosilane (Pierce) was added. The mixture was vortexed for 1 min and incubated for 30 min at 37° C. Samples were clarified by quick centrifugation and transferred to an Agilent 7683B autosampler. Sample analysis was randomized.

An Agilent 6890 gas chromatograph mated to a Waters GCT Premier time-of-flight mass spectrometer was used. Separation was effected using a 30 m×0.25 mm ID RTX-5Sil column with a film thickness of 0.25 μm. 1 μl derivatized sample was injected. A 10:1 split ratio was used. Inlet temperature was 250° C. Helium was used as the carrier gas (1 ml/min). The initial temperature was 85° C. and held for 1 min. Temperature was ramped at 8° C./min to 250° C. followed by a 16° C./min ramp to a final temperature of 330° C. The final temperature was held for 3.4 min. The mass spectrometer transfer line was set to 250° C.; the source temperature being 180° C. A 70 eV ion beam was employed with a trap current of 1.0 mA. Spectra were collected at a rate of 20 per sec with a mass range of 50-800 m/z. Data were collected using MassLynx 4.0 (Waters). Peak picking and deconvolution were performed using MarkerLynx and AMDIS 2.64 (National Institute of Standards and Technology). To identify possible markers the data were transferred to SIMCA-P+11.0 (Umetrics AB). Possible markers were analyzed in more detail by returning to the original chromatogram. Marker identification was performed using known standards or through analysis of the NIST database.

i. Oxidative Metabolism

WT and Oct1^{-/-} MEFs were plated in 24 well dishes at 1×10⁵ cells per well. Glucose oxidation was measured using D[U-¹⁴C]Glucose (Amersham), as described. For glutamate oxidation, similar conditions were used with L[U-¹⁴C]Glutamic acid (Amersham). Palmitate oxidation using [9,10(n)-³H]Palmitic acid (Amersham) was assayed as described.

j. RNAi

A549 cells expressing constitutive luciferase were infected with pRev-tet-ON (Clontech) to express the tetracycline transactivator. Scrambled and Oct1 shRNAs were cloned into MSCV-TMP vector (Open Biosystems).

k. Mitochondrial DNA

Real-time PCR was performed in quadruplicate using a Light cycler 480 (Roche) and SYBR Green (Invitrogen). Specificity of amplification and absence of primer dimers were confirmed by melting curve analysis.

l. TEM

WT and Oct1^{-/-} MEFs were grown on ACLAR. The cells were fixed in 2.5% glutaraldehyde/1% paraformaldehyde in 0.1M sodium cacodylate buffer, and post-fixied in 2% OsO₄, embedded in resin and sectioned. Sections were stained with uranyl acetate. Electron micrographs were taken using an FEI Philips Tecnai T-12.

m. ROS and Lactate

Male rag1^{-/-} C57BL/6 mice were repopulated with WT and Oct1^{-/-} embryonic livers as described. For ROS analysis, WBCs were harvested from spleens of recipient mice and stained with 5(6)-chloromethyl-2′7′-dichlorodihydrofluorescein diacetate-acetyl ester (CM-H₂DCFDA, Molecular Probes) for 30 min/37° C., stained on ice with anti-B220-PE and anti-thy-1-APC (BD biosciences), and analyzed by flow cytometry. Intracellular lactate was measured using a kit (Biovision).

n. ChIP in Oct1 Metabolic Assays

ChIP was performed as described previously except that SDS in the sonication buffer was adjusted to 0.2-0.5% and Protein G magnetic beads (ActivMotif) were used.

o. Calorimetery

6-8 week old WT and Oct1^{+/-}129 mice were fed normal (4.5%) or high fat (45%, Harlan) diets for 3 months, transferred to metabolic chambers, and studied for 72 hours using the Comprehensive Laboratory Animal Monitoring System (CLAMS; Columbus Instruments). Metabolic parameters were measured using a four-chamber open-circuit system. Respiratory exchange ratio (RER) was calculated as VCO₂/VO₂ normalized to the body weight. The basal metabolic rate was calculated using the formula metabolic rate=(3.815+1.232RER)×VO₂. Animals were acclimatized to the chambers prior to collecting data and maintained at 24° C. under a 12-hour light/dark cycle. Food and water were freely available. Mice were housed individually. 0.6 liters of air passed per minute. Each chamber was sampled for 1.5 minutes at 15-minute intervals. Exhaust O₂ and CO₂ content from each chamber was compared to ambient O₂ and CO₂ content. Food consumption was monitored by electronic scales; water by electronic sipper tubes and movement by XY/Z laser beam interruption.

p. Anchorage-Independent Growth

Assays were performed in six-well plates. The 2 ml top and bottom layers contained 0.3% and 0.6% Noble agar (Difco) in DMEM containing 10% FBS, 100 U/ml penicillin and 100 μg/ml streptomycin. MEFs were infected with H-RasV12 GFP virus (construct a gift of Chonghui Cheng). Infection efficiency was assayed by flow cytometry. MEFs were trypsinized, counted, plated in triplicate in the top layer and grown for 15 days at 37° C./5% CO₂. DCA was added to the media at the indicated concentrations. Colonies were stained with 0.005% crystal violet (Sigma). Images were analyzed by ImageJ software (NIH).

q. Mouse Survival/Tumor Spectrum

Oct1^{+/-}; p53^{+/-} mice were crossed to generate p53^{-/-} and Oct1^{+/-}; p53^{-/-} mice. Food and water were available ad lib. Mice were examined daily for any gross enlargement by palpation and for overall signs of discomfort. Mice were sacrificed if they developed ulcerated tumors or were moribund, or if the visible tumor mass was more than 1 cm in diameter.

r. Xenografts

2×10⁶ cells were injected subcutaneously in the flanks of female NCr nude mice (n=19) (Taconic). 2 mg/ml Doxycycline was added to the drinking water 24 hr post injection. The mice were monitored weekly using the IVIS® 100 system (Xenogen).

s. Statistics

Statistical analysis was performed using Microsoft Excel and Graph Pad Prism. The unpaired Student's t test was used to generate p values. Error bars depict±SEM. (*, p<0.05; **, p<0.01 and ***, p<0.001).

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

### C. SEQUENCES

```
  1. SEQ ID NO: 1
GGAATTAATTGCATGAATTAG
  2. SEQ ID NO: 2
GCCTTGAACCTCAGCTTTAAG
  3. SEQ ID NO: 3
TGTTACCAACTGGGACGACA
  4. SEQ ID NO: 4
CTATGGGAGAACGGCAGAAG
  5. SEQ ID NO: 5
AACATACGAAAAACACACCCATT
  6. SEQ ID NO: 6
AGTGTATGGCTAAGAAAAGACCTG
  7. SEQ ID NO: 7
CAGACCCCAGGTGGTACCGG
  8. SEQ ID NO: 8
TAACAGATGCACGGGGGTTG
  9. SEQ ID NO: 9
CTTCCCTGTTGAGTTGGCCC
10. SEQ ID NO: 10
CAAGCTGGAGCTAAATAAGC
11. SEQ ID NO: 11
ATCCCAGTTCACTTCTCTCCTG
12. SEQ ID NO: 12
GCAAACTAGAAGGCCTTAGAG
13. SEQ ID NO: 13
CTAATCTGGTATCCCCCGAGTCAC
14. SEQ ID NO: 14
CCGGGACACTTTTACATACATTTG
15. SEQ ID NO: 15
CCCTGCTCACATAATAACTCAAATC
16. SEQ ID NO: 16
GGGGCTACTTGGAAACCATTTC
17. SEQ ID NO: 17
CAATGGAAAGCGATTATAGCAACAAG
18. SEQ ID NO: 18
GGACTTCGCAGGCTCAGGCATAG
19. SEQ ID NO: 19
AACCACACCCACAGGATCAGA
20. SEQ ID NO: 20
TCTTCGCTTTATTGCTCCATGA
21. SEQ ID NO: 21
TGCTCCCCGGGCTGTAT
22. SEQ ID NO: 22
CATAGGAGTCCTTCTGACCCATTC
23. SEQ ID NO: 23
TGCTCCAGCATCGAATTTGTAC
24. SEQ ID NO: 24
AGAGCAGCTGCTGCTGCATACACTT
```

What is claimed is:

1. A method of identifying a cancer stem cell in a biological sample comprising assaying for the levels of Oct1 in the biological sample, wherein an increase in the amount of Oct1 in the biological sample as compared to a control is an indication of the presence of cancer stem cells in the biological sample.

2. The method of claim 1, wherein Oct1 levels are detected by immunohistochemistry.

3. The method of claim 1, wherein the biological sample is from a subject diagnosed with cancer.

4. The method of claim 2, wherein the biological sample comprises a tumor biopsy.

5. The method of claim 2, wherein the cancer comprises an adenocarcinoma.

6. The method of claim 5, wherein the adenocarcinoma comprises a colon adenocarcinoma, a breast carcinoma, or a lung carcinoma.

7. A method of treating cancer stem cells in a subject, comprising administering to the subject an inhibitor of Oct1 activity.

8. The method of claim 7, wherein the subject has been diagnosed as having cancer stem cells.

9. The method of claim 8, wherein the subject has been diagnosed as having cancer cells expressing high levels of Oct1 as compared to a control.

10. The method of claim 7, wherein the subject has undergone or been prescribed irradiation, chemotherapy, or a combination thereof.

11. A method of identifying an agent for use in treating a cancer stem cell comprising contacting a sample comprising Oct1 with a candidate agent and assaying for Oct1 activity in the sample, wherein a decrease in Oct1 activity in the sample is an indication that the candidate agent is an effective agent for use in treating cancer stem cells.

12. The method of claim 11, wherein the method comprises assaying for the ability of Oct1 to bind target DNA in the presence of the candidate agent, wherein a decrease in Oct1 binding to the target DNA is an indication that the candidate agent is an effective agent for use in treating cancer stem cells.

13. The method of claim 12, wherein the target DNA is the Oct1-binding site in the immediate promoter region of the Aldh1a1 promoter.
